SEQUENCE LISTING (136 sequences)

SEQ ID NO: 1 - DNA, 1490 bp, Artificial Sequence
  Rainbow Trout Ten M3 carboxy termini′
tccatctcgggggtgcaacaggaagtgacccggcaagccaaggctttcctgtccttcgagaggatgccggagatccagctgagccgccggcgctccaaccgggagaaaccctggctgtggttcgccaccgccaagtctctgatcggtaagggtgtcatgttggcggtgacgcagggccgtgtggtcaccaacgctctgaacatcgccaacgaggactgcatcaaggtcgccgccgtcctcaacaatgcgttctacctggaggacctgcacttcacggtggagggacgcgacacgcactacttcatcaagaccagcctcccggagagcgacctgggagcgctgaggctgacaagcgggaggaagtcgctggagaacggaagtcaacgtgactgtgtcccagtccaccaccgtggtgaacggcagaaccggcgcttcgccgacgtggagctgcagtacggcgctctagcgctccacgtgcgctatggcatgactctggacgaggagaaggcgcgtgtgctggagcaggccaggcagaaggcgttgtcgagtgcctggtccagggagcaacaacgggtgagggagggggaggagggggtgaggctgtggacggagggggagaagaggcagctgctgagcgggaggaaggttctgggctacgacgggtactacgtcctctccatagagcagtaccccgagctagcagactccgctaacaacatccagttcctcaggcagagcgaaatagggaagaggtaacagacagaatcctcggcactggccgccaaagagactaccccctccaaatcctgccccccaacctccctcgcctccccccttttctctaaaaagggggagggtccaggctagtgctgtgtttagcgccgactagctgaaacaaacagtaaaatgtagaatatcttaaactgaactatacctaatactaccactgtggggcctgaaaatcaaacaaaacggctccaactgacgcaaatgtttgtcccatgtgctatacagcgttgaatggactgtggactctcttgaaaagagagaaaaaaaagtcaaaactctcggtttgtgaaaggagaaaaaaacgttttttttttttttaaatagacttcctgaatttgctttcggaaaaaatattttaaaaagaaagaagaaatgtgtttacatacgcataacactacaacacgtctggactaatagaagaaaagccttctggtttcttacacaggacaacgtctataatctgattctacatcctgacgactgacctttgattgacctttgcgtactgaaaaaggtagtgttgttgttcgcagtaggaccatgggtctccaatggtggtaactagacagttaaaaccacttgttgaaaccacttgcttgttcttctgcttttctttccaaaagggacaaaacagctcccaccaagtgacttctttaccaatactagatcaaagtgggacgttttgggctcgtgccgaattc

SEQ ID NO: 2 - DNA, 756 bp, Artificial Sequence
  Rainbow Trout Ten M3 coding sequence of carboxy termini of Ten M3
tccatctcgggggtgcaacaggaagtgacccggcaagccaaggctttcctgtccttcgagaggatgccggagatccagctgagccgccggcgctccaaccgggagaaaccctggctgtggttcgccaccgccaagtctctgatcggtaagggtgtcatgttggcggtgacgcagggccgtgtggtcaccaacgctctgaacatcgccaacgaggactgcatcaaggtcgccgccgtcctcaacaatgcgttctacctggaggacctgcacttcacggtggagggacgcgacacgcactacttcatcaagaccagcctcccggagagcgacctgggagcgctgaggctgacaagcgggaggaagtcgctggagaacggaagtcaacgtgactgtgtcccagtccaccaccgtggtgaacggcagaaccggcgcttcgccgacgtggagctgcagtacggcgctctagcgctccacgtgcgctatggcatgactctggacgaggagaaggcgcgtgtgctggagcaggccaggcagaaggcgttgtcgagtgcctggtccagggagcaacaacgggtgagggagggggaggagggggtgaggctgtggacggagggggagaagaggcagctgctgagcgggaggaaggttctgggctacgacgggtactacgtcctctccatagagcagtaccccgagctagcagactccgctaacaacatccagttcctcaggcagagcgaaatagggaagaggtaa

SEQ ID NO: 3 - protein, 251 aa, Artificial Sequence
  Rainbow Trout Ten M3 carboxy termini of Ten M3
SISGVQQEVTRQAKAFLSFERMPEIQLSRRRSNREKPWLWFATAKSLIGKGVMLAVTQGRVVTNALNIANEDCIKVAAVLNNAFYLEDLHFTVEGRDTHYFIKTSLPESDLGALRLTSGRKSLENGVNVTVSQSTTVVNGRTRRFADVELQYGALALHVRYGMTLDEEKARVLEQARQKALSSAWSREQQRVREGEEGVRLWTEGEKRQLLSGRKVLGYDGYYVLSIEQYPELADSANNIQFLRQSEIGKR

SEQ ID NO: 4 - protein, 252 aa, Artificial Sequence
  Mouse Ten M1
MILGIQCELQKQLRNFISLDQLPMTPQYNEGRCLEGGKQPRFAAVPSVFGKGIKFAIKEGIVTADIIGVANEDSRRLAAILNNAHYLENLHFTIEGRDTHYFIKLGSLEEDLVLIGNTGGRRILENGVNVTVSQMTSVLNGRTRRFADIQLQHGALCFNIRYGTTVEEEKNHVLEMARQRAVAQAWTQEQRRLQEGEEGTRVWTEGEKQQLLGTGRVQGYDGYFVLSVEQYLELSDSANNIHFMRQSEIGRR

SEQ ID NO: 5 - protein, 253 aa, Artificial Sequence
  Mouse Ten M2
LITGVQQTTERHNQAFLALEGQVITKKLHASIREKAGHWFATTTPIIGKGIMFAIKEGRVTTGVSSIASEDSRKVASVLNNAYYLDKMHYSIEGKDTHYFVKIGAADGDLVTLGTTIGRKVLESGVNVTVSQPTLLVNGRTRRFTNIEFQYSTLLLSIRYGLTPDTLDEEKARVLDQAGQRALGTAWAKEQQKARDGREGSRLWTEGEKQQLLSTGRVQGYEGYYVLPVEQYPELADSSSNIQFLRQNEMGKR

SEQ ID NO: 6 - protein, 251 aa, Artificial Sequence
  Mouse Ten M3
PIFGVQQQVARQAKAFLSLGKMAEVQVSRRKAGAEQSWLWFATVKSLIGKGVMLAVSQGRVQTNVLNIANEDCIKVAAVLNNAFYLENLHFTIEGKDTHYFIKTTTPESDLGTLRLTSGRKALENGINVTVSQSTTVVNGRTRRFADVEMQFGALALHVRYGMTLDEEKARILEQARQRALARAWAREQQRVRDGEEGARLWTEGEKRQLLSAGKVQGYDGYYVLSVEQYPELADSANNIQFLRQSEIGKR

SEQ ID NO: 7 - protein, 243 aa, Artificial Sequence
  Mouse Ten M4
SILGVQCEVQKQLKAFVTLERFDQLYGSTITSCQQAPETKKFASSGSIFGKGVKFALKDGRVTTDIISVANEDGRRIAAILNNAHYLENLHFTIDGVDTHYFVKPGPSEGDLAILGLSGGRRTLENGVNVTVSQINTMLIQLQYRALCLNTRYGTTVDEEKVRVLELARQRAVRQAWAREQQRLREGEEGLRAWTDGEKQQVLNTGRVQGYDGFFVTSVEQYPELSDSANNIHFMRQSEMGRR

SEQ ID NO: 8 - protein, 252 aa, Artificial Sequence
  Human Ten M1
TILGIQCELQKQLRNFISLDQLPMTPRYNDGRCLEGGKQPRFAAVPSVFGKGIKFAIKDGIVTADIIGVANEDSRRLAAILNNAHYLENLHFTIEGRDTHYFIKLGSLEEDLVLIGNTGGRRILENGVNVTVSQMTSVLNGRTRRFADIQLQHGALCFNIRYGTTVEEEKNHVLEIARQRAVAQAWTKEQRRLQEGEEGIRAWTEGEKQQLLSTGRVQGYDGYFVLSVEQYLELSDSANNIHFMRQSEIGRR

SEQ ID NO: 9 - protein, 253 aa, Artificial Sequence
  Human Ten M2
LITGVQQTTERHNQAFMALEGQVITKKLHASIREKAGHWFATTTPIIGKGIMFAIKEGRVTTGVSSIASEDSRKVASVLNNAYYLDKMHYSIEGKDTHYFVKIGSADGDLVTLGTTIGRKVLESGVNVTVSQPTLLVNGRTRRFTNIEFQYSTLLLSIRYGLTPDTLDEEKARVLDQARQRALGTAWAKEQQKARDGREGSRLWTEGEKQQLLSTGRVQGYEGYYVLPVEQYPELADSSSNIQFLRQNEMGKR

SEQ ID NO: 10 - protein, 251 aa, Artificial Sequence
  Human Ten M3
PIFGVQQQVARQAKAFLSLGKMAEVQVSRRRAGGAQSWLWFATVKSLIGKGVMLAVSQGRVQTNVLNIANEDCIKVAAVLNNAFYLENLHFTIEGKDTHYFIKTTTPESDLGTLRLTSGRKALENGINVTVSQSTTVVNGRTRRFADVEMQFGALALHVRYGMTLDEEKARILEQARQRALARAWAREQQRVRDGEEGARLWTEGEKRQLLSAGKVQGYDGYYVLSVEQYPELADSANNIQFLRQSEIGRR

SEQ ID NO: 11 - protein, 252 aa, Artificial Sequence
  Human Ten M4
SILGVQCEVQKQLKAFVTLERFDQLYGSTITSCLQAPKTKKFASSGSVFGKGVKFALKDGRVTTDIISVANEDGRRVAAILNHAHYLENLHFTIDGVDTHYFVKPGPSEGDLAILGLSGGRRTLENGVNVTVSQINTVLSGRTRRYTDIQLQYGALCLNTRYGTTLDEEKARVLELARQRAVRQAWAREQQRLREGEEGLRAWTEGEKQQVLSTGRVQGYDGFFVISVEQYPELSDSANNIHFMRQSEMGRR

SEQ ID NO: 12 - protein, 252 aa, Artificial Sequence
  Zebrafish Ten M3
SISGVQQEVMRQAKAFLSFERMPEIQLSRRRSSREKPWLWFATVKSLIGKGVMLAITSKGQVATNALNIANEDCIKVVTVLNNAFYLEDLHFTVEGRDTHYFIKTSLPESDLGALRLTSGRKSLENGVNVTVSQSTTVVNGRTRRFADVELQYGALALHVRYGMTLDEEKARVLEQARQRALSSAWAREQQRVRDGEEGVRLWTEGEKRQLLSSGKVLGYDGYYVLSVEQYPELADSANNVQFLRQSEIGKR

SEQ ID NO: 13 - protein, 40 aa, Artificial Sequence
  Rainbow Trout TCAP3 (40a.a.)
QLLSGRKVLGYDGYYVLSIEQYPELADSANNIQFLRQSEI

SEQ ID NO: 14 - protein, 41 aa, Artificial Sequence
  Rainbow Trout TCAP 3 (41a.a.)
RQLLSGRKVLGYDGYYVLSIEQYPELADSANNIQFLRQSEI

SEQ ID NO: 15 - protein, 43 aa, Artificial Sequence
  Rainbow Trout preTCAP3 (43 a.a.)
QLLSGRKVLGYDGYYVLSIEQYPELADSANNIQFLRQSEIGKR

SEQ ID NO: 16 - protein, 44 aa, Artificial Sequence
  Rainbow Trout preTCAP3 (44 a.a.)
RQLLSGRKVLGYDGYYVLSIEQYPELADSANNIQFLRQSEIGKR

SEQ ID NO: 17 - DNA, 120 bp, Artificial Sequence
  Rainbow Trout TCAP3 (120 n.a.)
cagctgctgagcgggaggaaggttctgggctacgacgggtactacgtcctctccatagagcagtaccccgagctagcagactccgctaacaacatccagttcctcaggcagagcgaaata

SEQ ID NO: 18 - DNA, 123 bp, Artificial Sequence
  Rainbow Trout TCAP3 (123 n.a.)
aggcagctgctgagcgggaggaaggttctgggctacgacgggtactacgtcctctccatagagcagtaccccgagctagcagactccgctaacaacatccagttcctcaggcagagcgaaata

SEQ ID NO: 19 - DNA, 129 bp, Artificial Sequence
  Rainbow Trout preTCAP3 (129 n.a.)
cagctgctgagcgggaggaaggttctgggctacgacgggtactacgtcctctccatagagcagtaccccgagctagcagactccgctaacaacatccagttcctcaggcagagcgaaatagggaagagg

SEQ ID NO: 20 - DNA, 132 bp, Artificial Sequence
  Rainbow Trout preTCAP3 (132 n.a.)
aggcagctgctgagcgggaggaaggttctgggctacgacgggtactacgtcctctccatagagcagtaccccgagctagcagactccgctaacaacatccagttcctcaggcagagcgaaatagggaagagg

SEQ ID NO: 21 - protein, 40 aa, Artificial Sequence
  Zebrafish TCAP3 (40 a.a.)
QLLSSGKVLGYDGYYVLSVEQYPELADSANNVQFLRQSEI

SEQ ID NO: 22 - protein, 41 aa, Artificial Sequence
  Zebrafish TCAP3 (41 a.a.)
RQLLSSGKVLGYDGYYVLSVEQYPELADSANNVQFLRQSEI

SEQ ID NO: 23 - protein, 43 aa, Artificial Sequence
  Zebrafish preTCAP3 (43 a.a.)
QLLSSGKVLGYDGYYVLSVEQYPELADSANNVQFLRQSEIGKR

SEQ ID NO: 24 - protein, 44 aa, Artificial Sequence
  Zebrafish preTCAP3 (44 a.a.)
RQLLSSGKVLGYDGYYVLSVEQYPELADSANNVQFLRQSEIGKR

SEQ ID NO: 25 - DNA, 120 bp, Artificial Sequence
  Zebrafish TCAP3 (120 n.a.)
cagttgctcagctctgggaaggtgctgggttacgatggttactatgtactatcagtggagcaataccctgaactggccgacagtgccaacaatgtccagttcttgaggcagagtgagata

SEQ ID NO: 26 - DNA, 123 bp, Artificial Sequence
  Zebrafish TCAP3 (123 n.a.)
aggcagttgctcagctctgggaaggtgctgggttacgatggttactatgtactatcagtggagcaataccctgaactggccgacagtgccaacaatgtccagttcttgaggcagagtgagata

SEQ ID NO: 27 - DNA, 129 bp, Artificial Sequence
  Zebrafish TCAP3 (129 n.a.)
cagttgctcagctctgggaaggtgctgggttacgatggttactatgtactatcagtggagcaataccctgaactggccgacagtgccaacaatgtccagttcttgaggcagagtgagatagggaagagg

SEQ ID NO: 28 - DNA, 132 bp, Artificial Sequence
  Zebrafish preTCAP3 (132 n.a.)
aggcagttgctcagctctgggaaggtgctgggttacgatggttactatgtactatcagtggagcaataccctgaactggccgacagtgccaacaatgtccagttcttgaggcagagtgagatagggaagagg

SEQ ID NO: 29 - protein, 40 aa, Artificial Sequence
  Zebrafish TCAP4 (40 a.a.)
QLLSSGRVQGYEGFYIVSVDQFPELTDNINNVHFWRQTEM

SEQ ID NO: 30 - protein, 41 aa, Artificial Sequence
  Zebrafish TCAP4 (41 a.a.)
QQLLSSGRVQGYEGFYIVSVDQFPELTDNINNVHFWRQTEM

SEQ ID NO: 31 - protein, 43 aa, Artificial Sequence
  Zebrafish preTCAP4 (43 a.a.)
QLLSSGRVQGYEGFYIVSVDQFPELTDNINNVHFWRQTEMGRR

SEQ ID NO: 32 - protein, 44 aa, Artificial Sequence
  Zebrafish preTCAP4 (44 a.a.)
QQLLSSGRVQGYEGFYIVSVDQFPELTDNINNVHFWRQTEMGRR

SEQ ID NO: 33 - DNA, 120 bp, Artificial Sequence
  Zebrafish TCAP4 (120 n.a.)
cagctcctaagctctggacgtgtacagggctacgaaggcttctacatagtatcagtcgaccagttcccagagttgactgacaacataaataacgtccatttctggcgacagactgagatg

SEQ ID NO: 34 - DNA, 123 bp, Artificial Sequence
  Zebrafish TCAP4 (123 n.a.)
cagcagctcctaagctctggacgtgtacagggctacgaaggcttctacatagtatcagtcgaccagttcccagagttgactgacaacataaataacgtccatttctggcgacagactgagatg

SEQ ID NO: 35 - DNA, 129 bp, Artificial Sequence
  Zebrafish preTCAP4 (129 n.a.)
cagctcctaagctctggacgtgtacagggctacgaaggcttctacatagtatcagtcgaccagttcccagagttgactgacaacataaataacgtccatttctggcgacagactgagatgggacgcagg

SEQ ID NO: 36 - DNA, 132 bp, Artificial Sequence
  Zebrafish preTCAP4 (132 n.a.)
cagcagctcctaagctctggacgtgtacagggctacgaaggcttctacatagtatcagtcgaccagttcccagagttgactgacaacataaataacgtccatttctggcgacagactgagatgggacgcagg

SEQ ID NO: 37 - protein, 40 aa, Artificial Sequence
  Mouse TCAP1 (40 a.a.)
QLLGTGRVQGYDGYFVLSVEQYLELSDSANNIHFMRQSEI

SEQ ID NO: 38 - protein, 41 aa, Artificial Sequence
  Mouse TCAP1 (41 a.a.)
QQLLGTGRVQGYDGYFVLSVEQYLELSDSANNIHFMRQSEI

SEQ ID NO: 39 - protein, 43 aa, Artificial Sequence
  Mouse preTCAP1 (43 a.a.)
QLLGTGRVQGYDGYFVLSVEQYLELSDSANNIHFMRQSEIGRR

SEQ ID NO: 40 - protein, 44 aa, Artificial Sequence
  Mouse preTCAP1 (44 a.a.)
QQLLGTGRVQGYDGYFVLSVEQYLELSDSANNIHFMRQSEIGRR

SEQ ID NO: 41 - DNA, 120 bp, Artificial Sequence
  Mouse TCAP1 (120 n.a.)
cagcttttgggcaccgggagggtgcaggggtatgatgggtattttgtcttgtctgttgagcagtatttagaactttcagacagtgccaacaatattcacttcatgagacagagtgaaata

SEQ ID NO: 42 - DNA, 123 bp, Artificial Sequence
  Mouse TCAP1 (123 n.a.)
cagcagcttttgggcaccgggagggtgcaggggtatgatgggtattttgtcttgtctgttgagcagtatttagaactttcagacagtgccaacaatattcacttcatgagacagagtgaaata

SEQ ID NO: 43 - DNA, 129 bp, Artificial Sequence
  Mouse preTCAP1 (129 n.a.)
cagcttttgggcaccgggagggtgcaggggtatgatgggtattttgtcttgtctgttgagcagtatttagaactttcagacagtgccaacaatattcacttcatgagacagagtgaaataggcaggagg

SEQ ID NO: 44 - DNA, 132 bp, Artificial Sequence
  Mouse preTCAP1 (132 n.a.)
cagcagcttttgggcaccgggagggtgcaggggtatgatgggtattttgtcttgtctgttgagcagtatttagaactttcagacagtgccaacaatattcacttcatgagacagagtgaaataggcaggagg

SEQ ID NO: 45 - protein, 40 aa, Artificial Sequence
  Mouse TCAP2 (40 a.a.)
QLLSTGRVQGYEGYYVLPVEQYPELADSSSNIQFLRQNEI

SEQ ID NO: 46 - protein, 41 aa, Artificial Sequence
  Mouse TCAP2 (41 a.a.)
QQLLSTGRVQGYEGYYVLPVEQYPELADSSSNIQFLRQNEM

SEQ ID NO: 47 - protein, 43 aa, Artificial Sequence
  Mouse preTCAP2 (43 a.a)
QLLSTGRVQGYEGYYVLPVEQYPELADSSSNIQFLRQNEMGKR

SEQ ID NO: 48 - protein, 44 aa, Artificial Sequence
  Mouse preTCAP2 (44 a.a.)
QQLLSTGRVQGYEGYYVLPVEQYPELADSSSNIQFLRQNEMGKR

SEQ ID NO: 49 - DNA, 120 bp, Artificial Sequence
  Mouse TCAP2 (120 n.a.)
caactcctgagcacgggacgggtacaaggttatgagggctattacgtacttccggtggaacagtacccggagctggcagacagtagcagcaacatccagttcttaagacagaatgagagg

SEQ ID NO: 50 - DNA, 123 bp, Artificial Sequence
  Mouse TCAP 2 (123 n.a.)
cagcaactcctgagcacgggacgggtacaaggttatgagggctattacgtacttccggtggaacagtacccggagctggcagacagtagcagcaacatccagttcttaagacagaatgagatg

SEQ ID NO: 51 - DNA, 129 bp, Artificial Sequence
  Mouse preTCAP2 (129 n.a.)
caactcctgagcacgggacgggtacaaggttatgagggctattacgtacttccggtggaacagtacccggagctggcagacagtagcagcaacatccagttcttaagacagaatgagatgggaaagagg

SEQ ID NO: 52 - DNA, 132 bp, Artificial Sequence
  Mouse preTCAP2 (132 n.a.)
cagcaactcctgagcacgggacgggtacaaggttatgagggctattacgtacttccggtggaacagtacccggagctggcagacagtagcagcaacatccagttcttaagacagaatgagatgggaaagagg

SEQ ID NO: 53 - protein, 40 aa, Artificial Sequence
  Mouse TCAP3 (40 a.a.)
QLLSAGKVQGYDGYYVLSVEQYPELADSANNIQFLRQSEI

SEQ ID NO: 54 - protein, 41 aa, Artificial Sequence
  Mouse TCAP3 (41 a..a)
RQLLSAGKVQGYDGYYVLSVEQYPELADSANNIQFLRQSEI

SEQ ID NO: 55 - protein, 43 aa, Artificial Sequence
  Mouse preTCAP3 (43 a.a.)
QLLSAGKVQGYDGYYVLSVEQYPELADSANNIQFLRQSEIGKR

SEQ ID NO: 56 - protein, 44 aa, Artificial Sequence
  Mouse preTCAP3 (44 a.a.)
RQLLSAGKVQGYDGYYVLSVEQYPELADSANNIQFLRQSEIGKR

SEQ ID NO: 57 - DNA, 120 bp, Artificial Sequence
  Mouse TCAP3 (120 n.a.)
cagctgctgagcgctggcaaggtgcagggctacgatgggtactacgtactgtcggtggagcagtaccccgagctggctgacagtgccaacaacatccagttcttgcgacaaagtgagatc

SEQ ID NO: 58 - DNA, 123 bp, Artificial Sequence
  Mouse TCAP3 (123 n.a.)
cggcagctgctgagcgctggcaaggtgcagggctacgatgggtactacgtactgtcggtggagcagtaccccgagctggctgacagtgccaacaacatccagttcttgcgacaaagtgagatc

SEQ ID NO: 59 - DNA, 129 bp, Artificial Sequence
  Mouse preTCAP3 (129 n.a.)
cagctgctgagcgctggcaaggtgcagggctacgatgggtactacgtactgtcggtggagcagtaccccgagctggctgacagtgccaacaacatccagttcttgcgacaaagtgagatcggcaagagg

SEQ ID NO: 60 - DNA, 132 bp, Artificial Sequence
  Mouse preTCAP3 (132 n.a.)
cggcagctgctgagcgctggcaaggtgcagggctacgatgggtactacgtactgtcggtggagcagtaccccgagctggctgacagtgccaacaacatccagttcttgcgacaaagtgagatcggcaagagg

SEQ ID NO: 61 - protein, 40 aa, Artificial Sequence
  Mouse TCAP4 (40 a.a.)
QVLNTGRVQGYDGFFVTSVEQYPELSDSANNIHFMRQSEM

SEQ ID NO: 62 - protein, 41 aa, Artificial Sequence
  Mouse TCAP4 (41 a.a.)
QQVLNTGRVQGYDGFFVTSVEQYPELSDSANNIHFMRQSEM

SEQ ID NO: 63 - protein, 43 aa, Artificial Sequence
  Mouse preTCAP4 (43 a.a.)
QVLNTGRVQGYDGFFVTSVEQYPELSDSANNIHFMRQSEMGRR

SEQ ID NO: 64 - protein, 44 aa, Artificial Sequence
  Mouse preTCAP4 (44 a.a.)
QQVLNTGRVQGYDGFFVTSVEQYPELSDSANNIHFMRQSEMGRR

SEQ ID NO: 65 - DNA, 120 bp, Artificial Sequence
  Mouse TCAP4 (120 n.a.)
caggtgctgaacacggggcgggtgcaaggctacgacggcttctttgtgacctcggtcgagcagtacccagaactgtcagacagcgccaacaatatccacttcatgagacagagcgagatg

SEQ ID NO: 66 - DNA, 123 bp, Artificial Sequence
  Mouse TCAP4 (123 n.a.)
cagcaggtgctgaacacggggcgggtgcaaggctacgacggcttctttgtgacctcggtcgagcagtacccagaactgtcagacagcgccaacaatatccacttcatgagacagagcgagatg

SEQ ID NO: 67 - DNA, 129 bp, Artificial Sequence
  Mouse preTCAP4 (129 n.a.)
caggtgctgaacacggggcgggtgcaaggctacgacggcttctttgtgacctcggtcgagcagtacccagaactgtcagacagcgccaacaatatccacttcatgagacagagcgagatgggccgaagg

SEQ ID NO: 68 - DNA, 132 bp, Artificial Sequence
  Mouse preTCAP4 (132 n.a.)
cagcaggtgctgaacacggggcgggtgcaaggctacgacggcttctttgtgacctcggtcgagcagtacccagaactgtcagacagcgccaacaatatccacttcatgagacagagcgagatgggccgaagg

SEQ ID NO: 69 - protein, 40 aa, Artificial Sequence
  Human TCAP1 (40 a.a.)
QLLSTGRVQGYDGYFVLSVEQYLELSDSANNIHFMRQSEI

SEQ ID NO: 70 - protein, 41 aa, Artificial Sequence
  Human TCAP1 (41 a.a.)
QQLLSTGRVQGYDGYFVLSVEQYLELSDSANNIHFMRQSEI

SEQ ID NO: 71 - protein, 43 aa, Artificial Sequence
  Human preTCAP1 (43 a.a.)
QLLSTGRVQGYDGYFVLSVEQYLELSDSANNIHFMRQSEIGRR

SEQ ID NO: 72 - protein, 44 aa, Artificial Sequence
  Human preTCAP1 (44 a.a.)
QQLLSTGRVQGYDGYFVLSVEQYLELSDSANNIHFMRQSEIGRR

SEQ ID NO: 73 - DNA, 120 bp, Artificial Sequence
  Human TCAP1 (120 n.a.)
cagcttttgagcactgggcgggtacaaggttacgatgggtattttgttttgtctgttgagcagtatttagaactttctgacagtgccaataatattcactttatgagacagagcgaaata

SEQ ID NO: 74 - DNA, 123 bp, Artificial Sequence
  Human TCAP1 (123 n.a.)
cagcagcttttgagcactgggcgggtacaaggttacgatgggtattttgttttgtctgttgagcagtatttagaactttctgacagtgccaataatattcactttatgagacagagcgaaata

SEQ ID NO: 75 - DNA, 129 bp, Artificial Sequence
  Human preTCAP1 (129 n.a.)
cagcttttgagcactgggcgggtacaaggttacgatgggtattttgttttgtctgttgagcagtatttagaactttctgacagtgccaataatattcactttatgagacagagcgaaataggcaggagg

SEQ ID NO: 76 - DNA, 132 bp, Artificial Sequence
  Human preTCAP1 (132 n.a.)
cagcagcttttgagcactgggcgggtacaaggttacgatgggtattttgttttgtctgttgagcagtatttagaactttctgacagtgccaataatattcactttatgagacagagcgaaataggcaggagg

SEQ ID NO: 77 - protein, 40 aa, Artificial Sequence
  Human TCAP2 (40 a.a.)
QLLSTGRVQGYEGYYVLPVEQYPELADSSSNIQFLRQNEM

SEQ ID NO: 78 - protein, 41 aa, Artificial Sequence; identical to SEQ ID NO: 46
  Human preTCAP2 (41 a.a.)

SEQ ID NO: 79 - protein, 43 aa, Artificial Sequence; identical to SEQ ID NO: 47
  Human preTCAP2 (43 a.a.)

SEQ ID NO: 80 - protein, 44 aa, Artificial Sequence; identical to SEQ ID NO: 48
  Human preTCAP2 (44 a.a.)

SEQ ID NO: 81 - DNA, 120 bp, Artificial Sequence
  Human TCAP2 (120 n.a.)
cagcttctgagcaccgggcgcgtgcaagggtacgagggatattacgtgcttcccgtggagcaatacccagagcttgcagacagtagcagcaacatccagtttttaagacagaatgagatg

SEQ ID NO: 82 - DNA, 123 bp, Artificial Sequence
  Human TCAP2 (123 n.a.)
cagcagcttctgagcaccgggcgcgtgcaagggtacgagggatattacgtgcttcccgtggagcaatacccagagcttgcagacagtagcagcaacatccagtttttaagacagaatgagatg

SEQ ID NO: 83 - DNA, 129 bp, Artificial Sequence
  Human preTCAP2 (129 n.a.)
cagcttctgagcaccgggcgcgtgcaagggtacgagggatattacgtgcttcccgtggagcaatacccagagcttgcagacagtagcagcaacatccagtttttaagacagaatgagatgggaaagagg

SEQ ID NO: 84 - DNA, 132 bp, Artificial Sequence
  Human preTCAP2 (132 n.a.)
cagcagcttctgagcaccgggcgcgtgcaagggtacgagggatattacgtgcttcccgtggagcaatacccagagcttgcagacagtagcagcaacatccagtttttaagacagaatgagatgggaaagagg

SEQ ID NO: 85 - protein, 40 aa, Artificial Sequence; identical to SEQ ID NO: 53
  Human TCAP3 (40 a.a.)

SEQ ID NO: 86 - protein, 41 aa, Artificial Sequence; identical to SEQ ID NO: 54
  Human TCAP3 (41 a.a.)

SEQ ID NO: 87 - protein, 43 aa, Artificial Sequence
  Human preTCAP3 (43 a.a.)
QLLSAGKVQGYDGYYVLSVEQYPELADSANNIQFLRQSEIGRR

SEQ ID NO: 88 - protein, 44 aa, Artificial Sequence
  Human preTCAP3 (44 a.a.)
RQLLSAGKVQGYDGYYVLSVEQYPELADSANNIQFLRQSEIGRR

SEQ ID NO: 89 - DNA, 120 bp, Artificial Sequence
  Human TCAP3 (120 n.a.)
cagctgctgagcgccggcaaggtgcagggctacgacgggtactacgtactctcggtggagcagtaccccgagctggccgacagcgccaacaacatccagttcctgcggcagagcgagatc

SEQ ID NO: 90 - DNA, 123 bp, Artificial Sequence
  Human TCAP3 (123 n.a.)
cggcagctgctgagcgccggcaaggtgcagggctacgacgggtactacgtactctcggtggagcagtaccccgagctggccgacagcgccaacaacatccagttcctgcggcagagcgagatc

SEQ ID NO: 91 - DNA, 129 bp, Artificial Sequence
  Human preTCAP (129 n.a.)
cagctgctgagcgccggcaaggtgcagggctacgacgggtactacgtactctcggtggagcagtaccccgagctggccgacagcgccaacaacatccagttcctgcggcagagcgagatcggcaggagg

SEQ ID NO: 92 - DNA, 132 bp, Artificial Sequence
  Human preTCAP3 (132 n.a.)
cggcagctgctgagcgccggcaaggtgcagggctacgacgggtactacgtactctcggtggagcagtaccccgagctggccgacagcgccaacaacatccagttcctgcggcagagcgagatcggcaggagg

SEQ ID NO: 93 - protein, 40 aa, Artificial Sequence
  Human TCAP4 (40 a.a.)
QVLSTGRVQGYDGFFVISVEQYPELSDSANNIHFMRQSEM

SEQ ID NO: 94 - protein, 41 aa, Artificial Sequence
  Human TCAP4 (41 a.a.)
QQVLSTGRVQGYDGFFVISVEQYPELSDSANNIHFMRQSEM

SEQ ID NO: 95 - protein, 43 aa, Artificial Sequence
  Human preTCAP4 (43 a..a)
QVLSTGRVQGYDGFFVISVEQYPELSDSANNIHFMRQSEMGRR

SEQ ID NO: 96 - protein, 44 aa, Artificial Sequence
  Human preTCAP4 (44 a.a.)
QQVLSTGRVQGYDGFFVISVEQYPELSDSANNIHFMRQSEMGRR

SEQ ID NO: 97 - DNA, 120 bp, Artificial Sequence
  Human TCAP4 (120 n.a.)
caggtgctgagcacagggcgggtgcaaggctacgacggctttttcgtgatctctgtcgagcagtacccagaactgtcagacagcgccaacaacatccacttcatgagacagagcgagatg

SEQ ID NO: 98 - DNA, 123 bp, Artificial Sequence
  Human TCAP4 (123 n.a.)
cagcaggtgctgagcacagggcgggtgcaaggctacgacggctttttcgtgatctctgtcgagcagtacccagaactgtcagacagcgccaacaacatccacttcatgagacagagcgagatg

SEQ ID NO: 99 - DNA, 129 bp, Artificial Sequence
  Human preTCAP4 (129 n.a.)
caggtgctgagcacagggcgggtgcaaggctacgacggctttttcgtgatctctgtcgagcagtacccagaactgtcagacagcgccaacaacatccacttcatgagacagagcgagatgggccggagg

SEQ ID NO: 100 - DNA, 132 bp, Artificial Sequence
  Human preTCAP4 (132 n.a.)
cagcaggtgctgagcacagggcgggtgcaaggctacgacggctttttcgtgatctctgtcgagcagtacccagaactgtcagacagcgccaacaacatccacttcatgagacagagcgagatgggccggagg

SEQ ID NO: 101 - protein, 41 aa, Artificial Sequence
  G. gallus TCAP-1
QQLLNTGRVQGYDGYFVLSVEQYLELSDSANNIHFMRQSEI

SEQ ID NO: 102 - protein, 41 aa, Artificial Sequence; identical to SEQ ID NO: 30
  Zebrafish TCAP-4

SEQ ID NO: 103 - protein, 37 aa, Artificial Sequence
  D. melanogaster Ten-m gene product
ELVQHGDVDGWNGDIHSIHKYPQLADPGNVAFQRDAK

SEQ ID NO: 104 - protein, 41 aa, Artificial Sequence
  Human CRF TCAP like region
SEEPPISLDLTFHLLREVLEMARAEQLAQQAHSNRKLMEII

SEQ ID NO: 105 - protein, 40 aa, Artificial Sequence
  Human urocortin TCAP-like region
DNPSLSIDLTFHLLRTLLELARTQSQRERAEQNRIIFDSV

SEQ ID NO: 106 - protein, 38 aa, Artificial Sequence
  Human urocortin 2 TCAP-like region
IVLSLDVPIGLLQILLEQARARAAREQATTNARILARV

SEQ ID NO: 107 - protein, 38 aa, Artificial Sequence
  Human urocortin 3 TCAP=like region
FTLSLDVPTNIMNLLFNIAKAKNLRAQAAANAHLMAQI

SEQ ID NO: 108 - protein, 46 aa, Artificial Sequence
  L. migratoria DP
MGMGPSLSIVNPMDVLRQRLLLEIARRRLRDAEEQIKANKDFLQQI

SEQ ID NO: 109 - protein, 46 aa, Artificial Sequence
  A. domesticus DP
TGAQSLSIVAPLDVLRQRLMNELNRRRMRELQGSRIQQNRQLLTSI

SEQ ID NO: 110 - protein, 39 aa, Artificial Sequence
  T. molitor DP
SPTISITAPIDVLRKTWEQERARKQMVAQNNREFLNSLN

SEQ ID NO: 111 - protein, 41 aa, Artificial Sequence
  M. sexta DP-1
RMPSLSIDLPMSVLRQKLSLEKERKVHALRAAANRNFLNDI

SEQ ID NO: 112 - protein, 30 aa, Artificial Sequence
  M. sexta DP-II
SLSVNPAVDILQHRYMEKVAQNNRNFLNRV

SEQ ID NO: 113 - protein, 45 aa, Artificial Sequence
  P. Americana
TGSGPSLSIVNPLDVLRQRLLLEIARRRMRQSQDQIQNREILQTI

SEQ ID NO: 114 - protein, 41 aa, Artificial Sequence
  O. keta CRP
SDDPPISLDLTFHMLRQMNEMSRAEQLQQQAHSNRKMMEIF

SEQ ID NO: 115 - protein, 40 aa, Artificial Sequence
  R. norvegicus
DDPPLSIDLTFHLLRTLLELARTQSQRERAEQNRIIFDSV

SEQ ID NO: 116 - protein, 37 aa, Artificial Sequence
  P. sauvageii
QGPPISIDLSLELLRKMIEIEKQEKEKQQAANNRLLL

SEQ ID NO: 117 - protein, 41 aa, Artificial Sequence
  C. carpio US
NDDPPISIDLTFHLLRNMIEMARNENQREQAGLNRKYLDEV

SEQ ID NO: 118 - protein, 38 aa, Artificial Sequence
  M. Musculus UCN2
VILSLDVPIGLLRILLEQARYKAARNQAATNAQILAHV

SEQ ID NO: 119 - protein, 38 aa, Artificial Sequence
  R. dano UCN2
LTLSLDVPTNIMNVLFDVAKAKNLRAKAAENARLLAHI

SEQ ID NO: 120 - DNA, 305 bp, Artificial Sequence
  Hamster 305bp urocortin cDNA probe examples “cloning mRNA”
attcaccgccgctcgggatctgagcctgcaggcgagcggcagcgacgggaagaccttccgctgtccatcgacctcacattccacctgctacggaccctgctggagatggcccggacacagagccaacgcgagcgagcagagcagaaccgaatcatactcaacgcggtgggcaagtgatcggcccggtgtgggaccccaaaaggctcgaccctttcccctacctaccccggggctgaagtcacgcgaccgaagtcggcttagtcccgcggtgcagcgcctcccagagttaccctgaacaatcccgc

SEQ ID NO: 121 - DNA, 24 bp, Artificial Sequence
  TCAP1 fwd primer
acgtcagtgttgatgggaggacta

SEQ ID NO: 122 - DNA, 27 bp, Artificial Sequence
  TCAP1 rvs primer
cctcctgcctatttcactctgtctcat

SEQ ID NO: 123 - DNA, 25 bp, Artificial Sequence
  TCAP2 Fwd primer
tcgagggcaaggacacacactactt

SEQ ID NO: 124 - DNA, 26 bp, Artificial Sequence
  TCAP2 rvs primer
aagaactggatgttgctgctactgtc

SEQ ID NO: 125 - DNA, 25 bp, Artificial Sequence
  TCAP3 fwd primer
caacaacgccttctacctggagaac

SEQ ID NO: 126 - DNA, 21 bp, Artificial Sequence
  TCAP3 rvs primer
tgttgttggcactgtcagcca

SEQ ID NO: 127 - DNA, 23 bp, Artificial Sequence
  TCAP4 fwd primer
tttgcctccagtggttccatctt

SEQ ID NO: 128 - DNA, 24 bp, Artificial Sequence
  TCAP4 rvs primer
tggatattgttggcgctgtctgac

SEQ ID NO: 129 - protein, 6 aa, Artificial Sequence
  Conserved motif between CRF and TCAP I/L S X X (X)-L/V at amino terminus
XSXXXX

SEQ ID NO: 130 - protein, 4 aa, Artificial Sequence
  Conserved motif between CRF and TCAP - In middle  L/V-L/I-X-V/aliphatic residue
XXXX

SEQ ID NO: 131 - protein, 4 aa, Artificial Sequence
  Conserved motif between CRF and TCAP N/I/A-H/basic residue -I/L/F/-aliphatic at carboxy terminus
NXXX

SEQ ID NO: 132 - DNA, 8964 bp, Mus musculus
  exon (50)..(8197)
aagttctaagaagccggaccgatgtgcacagagaaggaatgaaggaagtatggatgtgaaggaacgcaggccttactgctccttgaccaagagcagacgggaaaaggaaaggcgctatacaaattcgtccgcggacaatgaggagtgtagggtccccacgcagaagtcctatagttccagtgaaaccttgaaagctttcgatcatgattattcacggctgctttatggaaacagagtaaaggatttggtccacagagaagccgacgagtatactagacaaggacagaattttaccctaaggcagttaggagtgtgtgaatccgcaactcgaagaggagtggcattctgtgcggaaatggggctccctcacagaggttactccatcagtgcagggtcagatgcggatacggaaaacgaagcagtgatgtcccctgagcatgccatgagactttggggcaggggggtcaaatcgggccgcagttcctgcctgtcaagccggtccaactccgccctcaccctgacagacacggagcacgagaacaggtcggacagtgagagcgagcaaccttcaaacaacccagggcaacccaccctgcagcctttgccgccatcccacaagcagcacccggcgcagcatcacccgtccatcacttccctcaatagaaactccctgaccaatagaaggaaccagagtccggccccgccggctgctttgcccgccgagctgcaaaccacacccgagtccgtccagctgcaggacagctgggtccttggcagtaatgtaccactggaaagcaggcatttcctattcaaaacagggacagggacgacgccactgttcagtacggcaaccccgggatacacaatggcatctggctctgtttattctccgcctacccggccacttcctagaaacaccctatcaagaagtgcttttaaattcaagaagtcttcaaagtactgcagctggaggtgcaccgcactgtgtgctgtaggggtctcagtgctcctggccattctcctctcctattttatagcaatgcatctatttggcctcaactggcacttacagcagacggaaaatgacacattcgagaatggaaaagtgaattctgacaccgtgccaacaaacactgtatcgttaccttctggcgacaatggaaaattaggtggatttacacatgaaaataacaccatagattccggagaacttgatattggccggagagcaattcaagaggttccccccgggatcttctggagatcgcagctctttattgatcagccacagtttcttaagttcaacatctctcttcagaaggatgcattgatcggagtgtacggccggaagggcttaccgccttcccatactcagtacgactttgtggaactactggatggtagcaggttaattgcgagagagcagcggaacctggtggagtccgaaagagccgggcggcaggcgagatctgtcagcctgcacgaagctggcttcatccagtacttggattctggaatctggcatctggctttttataacgacgggaaaaacccagagcaggtctcctttaacacgatcgttatagagtctgtggtggaatgcccccgaaattgccatggaaatggagagtgtgtttctggaacttgccattgtttccccgggtttctaggtccggattgttcaagagcagcctgtccggtgctctgtagtggcaacgggcaatactccaagggccgctgcctgtgcttcagtggctggaagggcaccgagtgtgacgtgccgacgacccagtgcattgacccgcagtgcgggggtcgtgggatttgcatcatgggctcttgcgcttgtaactcgggatacaaaggagaaaactgtgaggaagcggactgtctagaccctggatgttctaatcacggggtgtgtatccatggggaatgtcactgcaatccaggctggggtggcagcaactgtgaaatactgaagactatgtgtgcagaccagtgctcaggccacgggacttaccttcaagaaagcggctcctgcacttgcgacccaaattggactggccccgactgctcaaatgaaatatgttcagtggactgcggctcacacggcgtctgcatggggggctcctgtcgctgtgaagaaggctggaccggcccggcgtgtaatcagagagcttgccaccctcgctgtgctgagcacgggacgtgcaaggacggcaagtgcgagtgcagccaaggatggaacggagagcactgcacaattgctcactatttggataagatagttaaagagggttgccccggcttgtgcaacagcaatgggagatgcacactggaccaaaacggctggcactgcgtttgccagccagggtggagaggagcaggctgtgacgtagccatggagaccctctgtacagacagcaaagacaacgaaggagacggactcattgactgcatggatcctgattgctgcctccagagctcctgccaaaaccagccctactgtcgtggcttgcctgatcctcaggatatcattagccaaagccttcagacaccatctcagcaagctgccaagtccttctatgaccgaatcagtttcctgattggatcggatagcacccacgtgctccctggagaaagtccgttcaataagagtcttgcgtccgtcatcagaggccaagtactaacagctgatggaaccccacttattggcgtcaacgtgtcgtttttacactactcggaatatggatataccattacccgccaggatggaatgtttgacttggtggcaaatggtggcgcttctctgactttggtatttgagcgttccccattcctcactcagtaccacactgtgtggattccctggaatgtcttttatgtgatggatacccttgtcatgaagaaagaggagaacgacattcccagctgtgacctcagtggctttgtgaggccaagtcccatcattgtgtcttcaccgttatccaccttcttcaggtcttcccctgaggacagccccatcatccccgagacacaggtcctgcatgaagaaaccacaattccaggaacagatttgaaactttcctacctgagttccagagcggcagggtacaagtcagttcttaagattaccatgacccaggccgtcataccgtttaacctcatgaaggtccatctgatggtggccgtggttgggagactcttccagaagtggtttcctgcctcgccaaacttggcctacacgttcatctgggataagacggacgcatataatcagaaagtctacggcttgtcagaggcagttgtgtccgtcggatacgagtacgagtcgtgcttggacctgactctctgggaaaagaggactgccgttttgcaaggctatgagttggatgcttcgaacatgggcggctggacgttggacaagcaccatgtactggacgttcagaacggtatactatacaaaggaaatggagaaaatcagttcatctctcagcagcctccggtggtcagcagcatcatgggtaatggtcggaggcgtagcatctcatgcccaagttgcaatggtcaagctgacgggaacaaactcctggcacccgtggcgcttgcctgtgggatcgacggcagtctatacgtaggggatttcaattacgtccggcggatattcccgtctgggaatgtgacaagtgttttagaactaagaaataaagattttagacatagtagcaacccagctcacagatactacctggctacggacccagtcaccggagatttgtacgtctctgatactaacacccgcagaatctatcggccgaaatcactcacgggagccaaagacctgactaaaaacgctgaagtggtggcagggaccggggaacagtgccttccctttgacgaggccaggtgtggggatggaggcaaggctgtggaagcaacgctcatgagtcccaaaggaatggcaatcgataagaacggactgatctactttgttgatggaaccatgatcagaaaggttgatcaaaatggaatcatatcaactctcctgggctccaacgacctcacgtcagctcgacctttaacctgtgatactagcatgcatatcagccaggtgcgtctggaatggcccactgacctcgcgatcaaccccatggataactccatctacgtcctggataataacgtagttttacagatcactgaaaaccgtcaggtccgcatcgctgccgggcggcccatgcactgtcaggtccctggagtggaatacccggtggggaagcacgcggttcagaccaccctggagtcagccacggccattgctgtgtcctacagcggggtcctttacatcacggaaactgatgagaagaagatcaaccgaataaggcaggtcacgacagacggggagatctccttagtggctgggataccttcggaatgtgactgcaagaacgacgccaactgtgactgctaccaaagcggagacggctacgccaaagatgccaaactcaatgcgccgtcctccctggccgcctcgccagatggcactctgtacattgcagatctgggaaatatcaggatccgggccgtttcgaagaataaacctttactgaactcaatgaacttttacgaagttgcctctccaactgatcaagagctctacatctttgacatcaacggtactcaccagtacaccgtgagcctggtcacgggtgactacctatataattttagttacagcaatgacaatgacgtcaccgctgtaactgacagcaatggcaacaccctccgaatccgaagggatccgaatcggatgccggtgcgggtggtgtctcctgataaccaggtgatatggttgaccataggcaccaacgggtgtctgaaaagcatgaccgctcagggcctggaactggttttgtttacttaccatggcaacagtgggcttttagccaccaaaagtgacgaaactggatggacaacattttttgactatgacagtgaaggtcgcctgacgaatgttaccttccccactggggtggttacaaacctgcacggggacatggacaaggctatcacggtggacatcgagtcatccagcagagaggaagatgtcagcatcacttcgaacttgtcctccatcgattccttctacaccatggtccaagaccagttaagaaacagttaccagattgggtatgatggctcccttagaatcttctatgccagtggtctggactctcactaccagacagagccccacgttctggctggcacggcgaatcccacagtagccaaaagaaacatgactcttcccggtgagaacgggcagaatctggtggagtggagattccgaaaagaacaagcccagggcaaagtcaacgtattcggccggaagctcagggtcaatgggcgcaacctactctcagtggactttgatcggaccaccaagacggaaaagatctatgatgaccaccggaaatttctcctgaggatcgcttacgacacgtcggggcacccgactctctggctgccgagtagcaagctaatggcagtgaacgtcacctactcatccaccggtcaaattgccagcatccagagagggaccacgagcgaaaaggtggactatgacagccaggggaggatcgtatctcgggtctttgccgatgggaaaacatggagttacacgtacttggaaaagtccatggttcttctgctccatagccagcggcagtacatcttcgaatacgacatgtgggaccgcctgtccgccatcaccatgcccagtgtggctcgccacaccatgcagaccatccggtccattggctactaccgcaacatctacaatcccccagaaagcaatgcctctatcatcaccgactacaacgaggaagggctgcttctgcaaacagctttcctgggaacgagtcggagggtcttattcaagtatagaaggcagaccaggctatcagaaattttatacgacagcacaagagtcagttttacctacgacgaaacagcgggagtcctgaaaacagtaaaccttcagagtgatggttttatttgcaccattagatacaggcaaattggtcccctgattgacagacagattttccgcttcagcgaggatggaatggtaaatgcgagatttgactatagctacgacaacagctttcgagtgaccagcatgcagggtgtcatcaatgaaacaccactgcccattgatctataccagtttgatgacatctctggcaaagtcgagcagtttggaaaattcggagtgatatactacgacatcaaccaaatcatttccacggccgtgatgacttatacaaagcactttgatgctcatgggcgcatcaaggagatccaatatgagatatttaggtcactcatgtactggattacaattcaatatgataatatgggccgggtaaccaagagagagattaaaattgggccttttgccaacactaccaaatacgcgtacgagtacgacgtcgatggacagctccaaacagtttacctaaacgaaaagatcatgtggcggtacaactacgacctaaatggaaacctccacttgctcaaccccagcagcagcgcccgcctgacccctctgcgctatgacctgcgcgacagaatcacccgcctgggcgatgttcagtaccggctggatgaagatggtttcctgcgtcagaggggcactgaaatttttgaatacagctccaaagggcttctgactcgagtctacagtaaaggcagtggctggacagtgatctatcggtacgacggcctgggaagacgtgtttctagcaaaaccagcctgggacagcaccttcagtttttctacgccgacctgacataccccacgagaattactcacgtctacaaccattccagttcagaaatcacctccctgtactatgacctccaaggacatctcttcgccatggagatcagcagtggggatgagttctacatcgcctcggacaacacggggacaccgctggctgttttcagcagcaacgggctcatgctgaaacagacccagtacactgcctatggtgagatctactttgactccaacgtcgactttcagctggtaattggattccacgggggcttgtatgacccgctcaccaaactaatccactttggagaaagagattatgacattttggcgggaagatggaccacaccggacattgaaatctggaaaaggatcggaaaggaccctgctccttttaacctgtatatgtttcggaataacaaccccgcgagcaaaatccatgatgtgaaagattacatcacggatgttaacagctggctggtgacgtttggcttccatctgcacaatgctattcctggattccctgttcccaaatttgatttaactgagccttcctatgagcttgtgaagagtcaacagtgggaagatgtgccgcccatctttggagttcagcagcaagtggcaaggcaagccaaggccttcttgtccctggggaagatggccgaggtgcaggtgagccgacgcaaagctggcgccgagcagtcgtggctgtggttcgccacggtcaagtcgctcatcggcaagggcgtcatgctggccgtgagccaaggccgcgtgcagaccaacgtgctcaacatcgccaacgaggactgcatcaaggtggcggcggtgctcaacaacgccttctacctggagaacctgcacttcaccatcgagggcaaggacacacactacttcatcaagaccaccacacccgagagcgacctgggcacactgcggctgacgagcggtcgcaaggccctggagaacgggatcaacgtgaccgtgtctcagtccaccacggtggtgaacggcaggactcgcaggttcgccgacgtggagatgcagttcggtgccctggcactgcatgtgcgctatggcatgacgctggacgaggagaaggcgcgcattctggagcaggcgcgccagcgcgcgctcgcccgggcgtgggcacgggagcagcagcgcgtgcgcgacggcgaggagggtgcgcgcctctggacggagggtgagaaacggcagctgctgagcgctggcaaggtgcagggctacgatgggtactacgtactgtcggtggagcagtaccccgagctggctgacagtgccaacaacatccagttcttgcgacaaagtgagatcggcaagaggtaacccccgggccacccctgtgcagattctcctgtagcacaatccaaaccggactctccaaagagccttccaaaatgacactgctctgcagacagacacatcgcagatacacacgcaacacaaaccagaaacaaagacaacttttttttttttctgaatgaccttaaaggtgatcggctttaaagaatatgtttacatacgcatatcgctgcactcaattggactggaagtatgagaaaggaaaaaaaagcattaaaaaaggcaacgttttgccatgacccctctgtaccttcgaggcactgtatttaacaaaggttttaaaaaggaaaaaaaaatgcgtacaatgtttccagatattactgaattgtcgacctttgcttacaggaagtaatctctacttaggatgtgatatatatagatctgttcattttaaaatgtggggcaaagttactgtttatagaacccaactgctttcccgtgctgctttgtaaaaggacactggcacaagggacgtctgcttcggcggggatttaataatggattttactaacatggcttgccctgggagggaaaaactgacgaatagaatccttgtcactgataagcaaaggaaaccctgatttttttgtaaattatgtgagacaagttgtttatggatttttatatgaattacaatttactgtacatcaaatattagtctcagaggagttaatttatgtaaagtgtttaaaaagtttatacttaaaaataaaatgataaaaacaaaaaaa

SEQ ID NO: 133 - DNA, 2253 bp, Homo sapiens
  exon (107)..(1090)
gtgccccggatgtgcccagctggctcctggccccacccctcgggcctttgggctggaccagccacctctgcctgagacctccggtcgccgcaagaagctggagaggatgtacagcgttgaccgtgtgtctgacgacatccctattcgtacctggttccccaaggaaaatcttttcagcttccagacagcaaccacaactatgcaagcggtgttcaggggctacgcggagaggaagcgccggaaacgggagaatgattccgcgtctgtaatccagaggaacttccgcaaacacctgcgcatggtcggcagccggagggtgaaggcccagacgttcgctgagcggcgcgagcggagcttcagccggtcctggagcgaccccacccccatgaaagccgacacttcccacgactcccgagacagcagtgacctgcagagctcccactgcacgctggacgaggccttcgaggacctggactgggacactgagaagggcctggaggctgtggcctgcgacaccgaaggcttcgtgccaccaaaggtcatgctcatttcctccaaggtgcccaaggctgagtacatccccactatcatccgccgggatgacccctccatcatccccatcctctacgaccatgagcacgcaaccttcgaggacatccttgaggagatagagaggaagctgaacgtctaccacaagggagccaagatctggaaaatgctgattttctgccagggaggtcctggacacctctatctcctcaagaacaaggtggccacctttgccaaagtggagaaggaagaggacatgattcacttctggaagcggctgagccgcctgatgagcaaagtgaacccagagccgaacgtcatccacatcatgggctgctacattctggggaaccccaatggagagaagctgttccagaacctcaggaccctcatgactccttatagggtcaccttcgagtcacccctggagctctcagcccaagggaagcagatgatcgagacgtactttgacttccggttgtatcgcctgtggaagagccgccagcactcgaagctgctggactttgacgacgtcctgtgaggggcagaggcctccgcccagtcaccatcaggccactccctctgcaccgggacctggggctgggccgcctcgtgctccccgggactgtgtagctccggtctcgcctggagccacttcagggcacctcagacgttgctcaggttccccctgtgggttccggtcctcgctgcacccgtggccgcagaggctgcagtccctgggggccgggaggatcccgccctgtggcccgtggatgctcagcggccaggcactgacctgccatgcctcgcctggaggctcagctgtgggcatccctccatggggttcatagaaataagtgcaatttctacacccccgaaacaattcaaagggaagcagcatttcttgttaactagttaagcactatgctgctagttacagtgtaggcaccccggcccagcagcccagcagcccacatgtgttcaggaccctccctgcccaccccctccctgccgtatcgatcaccagcaccagggtggcccgtgtgcgtggggccagcgtcgccgggctgcccagcctggctctgtctacactggccgagtctctgggtctgtctacactggccgagtctccgactgtctgtgctttcacttacactcctcttgccaccccccatccctgcttacttagacctcagccggcgccggacccggtaggggcagtctgggcagcaggaaggaagggcgcagcgtcccctccttcagaggaggctctgggtggggcctgctcctcatccccccaagcccacccagcactctcattgctgctgttgagttcagcttttaccagcctcagtgtggaggctccatcccagcacacaggcctggggcttggcaggggcccagctggggctgggccctgggttttgagaaactcgctggcaccacagtgggcccctggacccggccgcgcagctggtggactgtaggggctcctgactgggcacaggagctcccagcttttgtccacggccagcaggatgggctgtcgtgtatatagctggggcgagggggcaggccccccttgtgcagagccaggggtctgagggcacctggctgtgttcccagctgagggagggctggggcgggggccgggcttggaacgatgtacgataccctcatagtgaccattaaacctgatcctcc

SEQ ID NO: 134 - DNA, 2253 bp, Danio rerio; identical to SEQ ID NO: 133
  exon (1)..(298)

SEQ ID NO: 135 - protein, 40 aa, Artificial Sequence
  TCAP 3 General Motif
QLLSXXKVXGYDGYYVLSXEQYPELADSANNXQFLRQSEI

SEQ ID NO: 136 - protein, 36 aa, Artificial Sequence
  G. gallus TCAP2
TGRVQGYEGYYVLPVEQYPELADSSSNIQFLRQNEM

(19) United States
(12) Patent Application Publication
(10) Pub. No.: US 2011/0288029 A1
(43) Pub. Date: Nov. 24, 2011

(54) METHOD FOR INHIBITING NEURONAL CELL DEATH

(76) Inventor: DAVID LOVEJOY, Stouffville (CA)

(21) Appl. No.: 13/197,575

(22) Filed: Aug. 3, 2011

Related U.S. Application Data
(63) Continuation of application No. 11/706,376, filed on Feb. 15, 2007, now abandoned.
(60) Provisional application No. 60/773,309, filed on Feb. 15, 2006.
(60) Provisional application No. 60/783,821, filed on Mar. 21, 2006.

(51) Int. Cl. A61K 38/17; A61P 25/28; A61P 25/16; C12N 5/0793; A61P 25/00

(52) U.S. Cl. 514/17.7; 435/375

(57) ABSTRACT

This invention relates to a method of inhibiting neuronal cell death, including protecting neronal cells from cell death and the effects of stress, such as high or low pH, comprising administering to the cells an effective amount of Teneurin C Associate Peptide (TCAP). The invention provides the use of TCAP to prevent and/or treat a number of brain conditions, such as hypoxia-ischemia and brain alkalosis. In another aspect, the invention provides a method of treating various pH induced neuronal conditions.

### RELATED APPLICATIONS

This application is a continuation of U.S. patent application Ser. No. 11/170,376, filed Feb. 15, 2007, entitled “A Method for Inhibiting Neuronal Cell Death” which claims the benefit and priority of U.S. provisional application No. 60/773,309, filed Feb. 15, 2006, entitled “A Method for Inhibiting Neuronal Cell Death” and U.S. 60/783,821, filed Mar. 21, 2006, entitled “Method for Regulating Neurite Growth”. All of these references are incorporated in their entirety be reference.

### FIELD OF THE INVENTION

This invention relates to a method for inhibiting neuronal cell death. It further relates to the neuroprotective effects of teneurin C-terminal associate peptides (TCAP) and to methods and uses of TCAP as a neuroprotective agent and/or to inhibit neuronal cell death. In one aspect it relates to the uses of TCAP to inhibit pH stress-induced neuronal cell death.

### BACKGROUND OF THE INVENTION

The teneurins are a family of four vertebrate type II transmembrane proteins preferentially expressed in the central nervous system (Baumgartner et al., 1994). The teneurins are about 2800 amino acids long and possess a short membrane spanning region. The extracellular face consists of a number of structurally distinct domains suggesting that the protein may possess a number of distinct functions (Minet and Chiquet-Ehrismann, 2000; Minet et al., 1999; Oohashi et al., 1999). The gene was originally discovered in Drosophila as a pair rule gene and was named tenascin-major (Ten-M) or Odz (Baumgartner et al., 1994; Levine et al., 1994). It is expressed in the Drosophila nervous system and targeted disruption of the genes leads to embryonic lethality (Baumgartner et al., 1994). In immortalized mouse cells, expression of the teneurin protein led to increased neurite outgrowth (Rubin et al., 1999).

The extracellular C-terminal region of each teneurin is characterized by a 40 or 41 amino acid sequence flanked by enzymatic cleavage sites, which predicts the presence of an amidated cleaved peptide (Qian et al., 2004; Wang et al., 2005). A synthetic version of this peptide was named teneurin C-terminus associated peptide (TCAP) and is active in vivo and in vitro. The mouse TCAP from teneurin-1 (TCAP-1) can modulate cAMP concentrations and proliferation in mouse hypothalamic cell lines as well as regulate the teneurin protein in a dose dependent manner (Wang et al, 2004). Intracerebroventricular injection of TCAP-1 into rats can induce changes in the acoustic startle response three weeks after administration (Wang et al., 2005). [Also see, PCT/CA2003/000622, filed May 2, 2003, published Nov. 13, 2003, herein incorporated by reference.]

Currently, it is thought that following initial trauma, neurons die by necrosis, apoptosis or a combination of the two (Thompson, 1995; Columbano., 1995; Rosser and Gores, 1995; Watson, 1995). Necrosis has been defined as unprogrammed cell death induced by physiological trauma, such as hypoxia, injury, infection and cancer. The role of pH in the brain during these times of stress depends upon the trauma inflicted as both phenomenon can occur simultaneously depending upon pathological conditions, physiological activators, physical trauma, environmental toxins and carcinogenic chemicals (Wyllie et al., 1980; Arends and Willie, 1991; Buja et al., 1993; Majno and Jorris, 1995). Various neurodegenerative diseases, such as brain ischemia and Huntington's Disease, exist contingent upon various forms of cell death that, in turn, are mediated by their environments' surrounding pH. Although extracellular pH changes under normal metabolic circumstances, a number of pathological conditions affect pH and lead to cell death.

One of the logistical problems in understanding cell death and its corroborating factors is the ambiguity surrounding cell death. The current research indicates that many characteristics that were once thought to pertain only to apoptosis, now apply to necrosis as well. The current consensus is that following the initial insult such as during brain ischemia, brain cells die by necrosis, apoptosis or a combination of the two and pH plays a pivotal role during these times, specifically alkaline pH (Levine et al., 1992; Robertson, 2002).

Although, the literature on brain acidosis is extensive, brain alkalosis is not well understood (Robertson, 2002). Intracellular alkalinization has been observed in cells undergoing cytokine deprivation (Khaled, 1999) as well as hypoxia-ischemia (HI) (Robertson, 2002). For example, during brain ischemia, brain pH levels indicated a progression from early acidosis to subacute alkalosis (Levine et al., 1992).

There is a need to counteract the effects of stress, such as pH induced cellular stress on the brain and to develop methods and compounds to protect cells against said effects, accordingly.

### SUMMARY OF THE INVENTION

In one aspect the invention provides a method for inhibiting neuronal cells against cell death. The inventors have surprisingly found that TCAP treated cells survive better in stress conditions, for instance in pH induced stress conditions, and in one aspect in alkaline pH conditions compared to vehicle treated cells.

As such, in one aspect the invention provides a method for inhibiting neuronal cells against cell death by administering an effective amount of TCAP, pharmaceutically acceptable salt or ester thereof or obvious chemical equivalent thereof to the cells. In another embodiment, administration of TCAP to the cells is administration of TCAP to a patient in need thereof comprising said cells. In one aspect the patient in need thereof is a patient who sustained or is suspected to have sustained a physiological trauma In one aspect, a pharmaceutical composition comprising TCAP, pharmaceutically acceptable salt or ester or obvious chemical equivalent thereof and a pharmaceutically acceptable carrier is administered.

In one aspect, the invention provides a method of inhibiting and/or preventing neuronal cell death comprising administering to the cell an effective amount of TCAP, a pharmaceutical acceptable salt or ester thereof or obvious chemical equivalent thereof.

In one embodiment, inhibiting neuronal cell death comprises inhibiting and/or protecting and/or preventing neuronal cells from cell death under conditions where cell death may occur, such as a result of physiological trauma.

In one embodiment, conditions wherein cell death may occur are conditions conducive to necrosis. As such, in one aspect the invention provides a method of inhibiting, preventing or protecting neuronal cells from cell death by necrosis by administering an effective amount of TCAP, pharmaceutically acceptable salt or ester thereof or obvious chemical equivalent thereof.

In one embodiment, conditions where cell death may occur is stress-induced neuronal cell death, such as pH-induced neuronal cell death. In one aspect, pH-induced neuronal cell death is alkalosis-stress induced neuronal cell death or cell death as a result of high pH conditions. In one aspect, high pH conditions are conditions wherein pH is greater than 7.4. In another aspect, the pH is 8.0 or greater. In another aspect, the pH is from 8.0 to 9.0, 8.0 to 8.5, or 8.0 to 8.4. In another aspect, one condition of pH induced stress is from 6.0 to 7.4 or at pH 6.8.

In another aspect, the physiological trauma is selected from the group consisting of: hypoxia, injury, infection, cytokine deprivation, carcinogenic agents and cancer and/or is related to or the result of a neurodegenerative disease.

In one aspect, the neurodegenerative disease is selected from the group consisting of: Alzheimer's, Parkinson's, Huntington's, Multiple Sclerosis and brain ischemia.

In yet another embodiment, the physiological trauma is selected from the group consisting of: hypothermia, hypoxia, acute ischemia, hypoxia-ischemia, respiratory alkalosis, metabolic alkalosis and brain alkalosis. In another embodiment, it is traumatic injury to the brain or spinal cord or a result of secondary energy failure post the physiological trauma.

In one embodiment, the invention provides a method for using an effective amount of TCAP, pharmaceutical acceptable salt or ester thereof or obvious chemical equivalent thereof in the treatment of a neuronal condition associated with alkaline neuronal cell pH, by administering said TCAP to the patient in need thereof. In one aspect said condition is related to pH conditions greater than 7.4, 8.0 or greater, from 8.0 to 9.0, or from 8.0 to 8.4.

In one embodiment of the aforementioned methods of the invention, the neuronal cell is an immortalized mouse hypothalamic cell.

In one embodiment, the invention provides a method of screening of modulators of the neuronal cell death inhibitory effects of TCAP, comprising administering TCAP to neuronal cells under conditions that would normally induce neuronal cell death if TCAP were not present (e.g. pH induced cell death, alkalosis induced cell death); administering a suspected modulator of said TCAP function and determining the effects of said suspected modulator on TCAP inhibition of neuronal cell death. If said suspected modulator enhances TCAP inhibition of neuronal cell death or decreases TCAP inhibition of neuronal cell death, then it is a modulator of TCAP inhibition of neuronal cell death. In one embodiment, said suspected modulator is administered to the cells prior to, simultaneously with and/or after administration of TCAP. In another embodiment, determining the effects of said modulator comprises comparing the levels of neuronal cell death and/or survival with a control, such as cell death absent the presence of TCAP or modulator; in the presence of TCAP alone or modulator alone, or compared to established baseline effects of neuronal cell death under various conditions.

In another aspect of the invention, the invention provides a method for increasing neuronal cell proliferation under conditions of neutral pH or acidosis pH conditions. In one embodiment, the pH conditions are pH of 7.4 or less. In another embodiment, the pH conditions are 6.8 or less. In yet another embodiment the pH conditions are between 6.8 and 7.4.

Additional aspects and advantages of the present invention will be apparent in view of the description which follows. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The invention will now be described in relation to the drawings, in which:

FIG. 1a. Cell morphology of N38 cells at 48 hours as a function of pH treatment.

FIG. 1b. Cell morphology of N38 cells at 72 hours as a function of pH treatment.

FIG. 1c. Example of a necrotic cell.

FIG. 1d. Quantification of small crenated (necrotic) cells. The levels of significance were determined by two-way ANOVA using Bonferroni's Post Test.

FIG. 2a. Proliferation of N38 cells as a function of pH. TCAP-1 (2×10⁻⁷ M) increased the number of cells post 48 hrs after treatment at pH extremes 6.8, 8.0, 8.4. The level of significance was determined using a two-way analysis of variance (ANOVA).

FIG. 2b. Changes in cell viability over 48 hours as determined by trypan blue uptake. TCAP increased the number of viable cells at pH 6.8 (p<0.10) pH 8.0 (p<0.001) and pH 8.4 (p<0.05). The level of significance was determined using a two-way analysis of variance (ANOVA).

FIG. 3. Changes in mitochrondrial metabolism of N38 cells as determined by the MTT assay. TCAP-1 (2×10⁻⁷ M) increased the number of viable cells post 48 hrs after treatment at pH extremes 8.0 and 8.4. The level of significance was determined using a two-way analysis of variance (ANOVA).

FIG. 4a. Apoptotic, necrotic and healthy cells fluorescent microscopy quantification analyses post 48 hrs. Cell types are characterized by colour: apoptosis (green) necrosis (red) healthy (blue).

FIG. 4b. Example of apoptotic cell.

FIG. 4c. Apoptotic, necrotic and healthy cells fluorescent microscopy quantification analyses. TCAP significantly decreased the amount of necrotic cells post 48 hrs at pH extremes 6.8 (P<0.0001), 8.0 (P<0.0001), 8.4 (P<0.0001). A two way ANOVA was used to determine levels of significance.

FIG. 5a. Caspase 8 colorimetric assay at pH extremes.

FIG. 5b. Caspase 3 colorimetric assay at pH extremes.

FIG. 5c. Caspase 3 western blot.

FIG. 6a. PARP quantification using transformed data.

FIG. 6b. PARP western blot detection at pH extremes. Post 48 hrs TCAP-1 (2×10⁻⁷ M).

FIG. 6c, PARP optical density quantification.

FIG. 7a. Akt quantification using transformed data.

FIG. 7b. Akt western blot detection at pH extremes. Post 48 hrs TCAP-1 (2×10⁻⁷ M).

FIG. 7c. Phospho-Akt western blot detection at pH extremes. There was no indication of AKT phosphorylation in any sample except for the control, thus TCAP is not rescuing cells through the AKT cell survival pathway.

FIG. 8. BrdU colorimetric assay at pH extremes.

### DETAILED DESCRIPTION OF THE INVENTION

As described herein, teneurin C-terminus associated peptide (TCAP) inhibits neuronal cell death, such as during timed of pH induced cellular stress in the brain. In another aspect, TCAP has a neuroprotective effect, protecting neuronal cells from cell death, such as, during times of pH induced cellular stress in the brain. In one aspect of the invention, such pH induced cellular stress in the brain is related to hypoxia-ischemia and/or brain alkalosis. In the examples described herein, an immortalized hypothalamic mouse cell line (N38) was treated with medium buffered at pHs 6.8, 7.4, 8.0 and 8.4 treated with 100 nM TCAP and examined at 24 and 48 hours. TCAP significantly increased cell proliferation at pH 6.8 and inhibited declines in cell proliferation at pHs 8.0 and 8.4 as determined by direct cell viability assays. TCAP did not significantly alter caspase 8 and 3 activity, nor induce PARP cleavage. TCAPs effects on the S phase of cell cycling were investigated through a Bromodeoxyuridine (BrdU), the results showing that TCAP does not have a major effect during the S phase of cell proliferation. The incidence of necrosis was tested via cell viability (Trypan Blue) assay and fluorescence microscopy utilizing fluorophores to Annexin V, Ethidium Homodimer III and Hoechst as well as morphology analyses. The results indicate that TCAP can protect cells from necrosis. In one aspect, TCAP has a neuroprotective role during times of cellular stress, such as induced pH stress. As such, TCAP can be used in the treatment of physiological effects of pH in the brain during trauma, such as hypoxia-ischemia.

### DEFINITIONS

“Administering to the cell(s)” as used herein means both in vitro and in vivo administration to the cells and can be direct or indirect administration, as long as the cells are at some point exposed to the substance being administered.

“Apoptosis” as used herein means “programmed cell death” and is a necessary event of normal development. It is a normal process for eliminating unwanted cells.

“Effective Amount” and “Therapeutically Effective Amount” as used herein means an amount effective, at dosages and for periods of time necessary to achieve the desired results. For example, an effective amount of a substance may vary according to factors such as the disease state, age, sex, and weight of the individual, and the ability of the substance to elicit a desired response in the individual. Dosage regimes may be adjusted to provide the optimum therapeutic response. For example, several divided doses may be administered daily or the dose may be proportionally reduced as indicated by the exigencies of the therapeutic situation.

“Homeostasis” as used herein means the inherent tendency in an organism or cell toward maintenance of physiological stability and making automatic adjustments in relation to its environment. Other wise known as normal stability of the internal environment (Sapolsky, 1992).

“Inhibiting Neuronal Cell Death” as used herein include inhibiting, preventing, and protecting neuronal cells (including rescuing neuronal cells) from, cell death.

“Necrosis” as used herein means unprogrammed cell death induced by physiological trauma, such as hypoxia, injury, infection and cancer/carcinogenic agents.

“Neuronal Cells” as used herein includes, but is not limited to, immortalized mouse hypothalamic neurons.

“Obvious Chemical Equivalents” as used herein means, in the case of TCAP, any variant that does not have a material effect upon the way the invention works and would be known to a person skilled in the art. For instance, this could include but not necessarily be limited to any salts, esters, conjugated molecules comprising TCAP, truncations or additions to TCAP.

“Pharmaceutically Acceptable Carrier” as used herein means any medium which does not interfere with the effectiveness or activity of an active ingredient and which is not toxic to the hosts to which it is administered. It includes any carrier, excipient, or vehicle, which further includes diluents, binders, adhesives, lubricants, disintegrates, bulking agents, wetting or emulsifying agents, pH buffering agents, and miscellaneous materials such as absorbants that may be needed in order to prepare a particular composition. Examples of carriers, excipient or vehicles include but are not limited to saline, buffered saline, dextrose, water, glycerol, ethanol, and combinations thereof. The use of such media and agents for an active substance is well known in the art (e.g., “Remington: The Sciences and Practice of Pharmacy, 21^{st} Edition”, (University of the Sciences in Philadelphia, 2005).

“Neuronal condition associated with alkaline neuronal cell pH” as used herein means any neuronal condition that is caused by or causes or results in or is associated with alkaline neuronal cell pH. Such conditions include, but are not limited to brain ischemia, neurodegenerative diseases such as, Alzheimer's, Parkinson's, Huntington's, brain ischemia and multiple sclerosis, and brain injury associated with physiological trauma.

“Stressor” is defined as anything that disrupts physiological balance, be it physical or psychological (Sapolsky, 1992).

“Stress-related brain or neuronal condition” as used herein means any brain neuronal condition associated with neuronal cells not being in a state of homeostasis.

“TCAP” as used herein means a 38-41 amino acid sequence, or in one embodiment a 40-41 amino acid sequence, from the C-terminal end of a teneurin peptide and all analogs, homologs, fragments, derivatives, salts, esters of the TCAP peptide which have the desired activity, and obvious chemical equivalents thereto, as described in PCT/CA2003/000622, filed May 2, 2003, published Nov. 13, 2003, and which is herein incorporated by reference. For instance, in one embodiment, TCAP includes human or mouse TCAP, such as TCAP-1, such as SEQ. ID. NOs. 37-44 (mouse) or 69-76 (human) of PCT/CA2003/000622 and analogs, homologs, fragments, derivatives, salts, esters and obvious chemical equivalents thereof. In one embodiment the TCAP is mouse TCAP-1 having the amino acid sequence: QQLLGTGRVQGYDGYFVLSVEQYLELSDSANNIHFMRQSEI-NH2 (accession number nm 011855 (SEQ. ID. NO. 38)). In one embodiment TCAP is prepared by solid phase synthesis and stored as a lyophilized powder at −80° C. reconstituted by alkalinizing with ammonium hydroxide and dissolved into physiological saline at 10⁻⁴ M stock solution.

“A nucleotide encoding TCAP” as used herein means a nucleotide sequence that encodes TCAP, including DNA and RNA. Such suitable sequences are described in PCT/CA2003/000622, which is herein incorporated by reference.

### Applications: The Use of TCAP to Inhibit Neuronal Cell Death

The invention broadly contemplates the use of TCAP, including an isolated TCAP, a nucleotide encoding TCAP to inhibit neuronal cell death.

### (a) Necrosis in Neurodegenerative Diseases

Necrotic cell death in the central nervous system follows acute ischemia or traumatic injury to the brain or spinal cord (Linnik, 1993; Emery, 1998). It occurs in areas that are most severely affected by abrupt biochemical collapse, which leads to the generation of free radicals and excitotoxins (e.g., glutamate, cytotoxic cytokines, and calcium). The histologic features of necrotic cell death are mitochondrial and nuclear swelling, dissolution of organelles, and condensation of chromatin around the nucleus. These events are followed by the rupture of nuclear and cytoplasmic membranes and the degradation of DNA by random enzymatic cuts in the molecule (Martin, 2001). Given these mechanisms and the rapidity with which the process occurs, necrotic cell death is extremely difficult to treat or prevent. The present inventors herein describe a method of treating and/or preventing necrotic cell death using TCAP.

### (b) pH in Necrosis

According to Potapenko et al., brain alkalinization induces an increase of Ca²⁺ in neurons due to Ca²⁺ sequestering structures, such as the mitochondria and endoplasmic reticulum, and elevated cytoplasmic Ca²⁺ is implicated in neuronal cell death, more specifically, necrosis during brain ischemia (Yuan et al., 2003). As mentioned previously such excessive rises in Ca²⁺ may be induced by excitoxicity caused by brain ischemia, subsequently overstimulating postsynaptic glutamate receptors; of these glutamate-gated channels, NMDA receptor channels play a key role in excitotoxicity as they conduct both Na⁺ and Ca²⁺ (Bonfoco et al. 1995).

### (c) Brain Injuries Related to Alkalosis

Insults to the brain can quite often lead to shifts in pH and based on the data presented it appears that TCAP is rescuing neurons from necrosis consistently at high pH extremes, such as pH 8.0 and 8.4. Dying neurons are a clear indication of many neurodegenerative diseases including Alzheimer's, Parkinson's, Huntington's, brain ischemia and multiple sclerosis (Siao, 2002). These neurodegenerative conditions are characterized by their deleterious effects on brain function resulting from deterioration of neurons. The destruction of neurons in these conditions may be regulated by various forms of cell death and can be caused by damaged mitochondrion, increased levels of excitotoxins such as glutamate, which increases calcium influx into the neurons and activates calcium dependent enzymes such as calpain and caspases (Randall & Thayer, 1992; Brorson et al., 1995) and pH. Brain pH during times of neurodegenerative stress is not well understood, however, calcium and pH are not mutually exclusive, during both respiratory and metabolic alkalosis, increases in calcium occur in rat neurons due to intracellular calcium accumulating structures such as the mitochondrion (Potapenko, 2004), this is also substantiated by the fact that glutamate induced neuron death requires mitochondrial calcium uptake (Stout et al., 1998).

Recent studies on brain energy metabolism using phosphorous and proton magnetic resonance (MR) spectroscopy have allowed an understanding of energy changes within the brain following (HI) (Thornton, 1998; Moon, 1973). A phenomenon named the “secondary energy failure” that occurs some 8-24 hours after the initial insult has been recently discovered, and have correlated the magnitude of this disruption with the eventual neurodevelopmental outcome (Thornton, 1998). A similar relationship between intracellular alkalosis and the severity of brain injury in infants has also found that babies with the most alkaline brain cells had more severe changes on MR imaging within the first 2 weeks of life and the worst neurodevelopmental outcome at one year (Roberstson, 2002). Thus, a means of identifying neuropeptides with pH protective properties would be a pivotal finding as it would provide novel therapeutic treatments. The inventors have shown herein that TCAP is a neuroprotective peptide and can inhibit neuronal cell death. As such, it can be used to treat a number of neuronal conditions, such as a neuronal condition associated with alkaline neuronal cell pH.

### (d) Neuronal Cell Death Inhibition/Neuroprotective Role of TCAP During Times of Stress

The potential for neuropeptides to regulate brain processes during times of stress (e.g. as a result of a stress-related brain or neuronal condition) is an important paradigm in the search for novel ways of coping with neurodegenerative diseases and physiological stress and examples of neuropeptides being connected with therapeutic uses are plentiful. (Oozes et al., 1994; Glazer et al. 1994; Zhang et al., 2001).

The teneurin C-terminus associated peptides (TCAP) have a neuroprotective effect from cell death, during times of pH induced cellular stress in the brain such as during hypoxia-ischemia. The present inventors herein describe a method of treatment or use of TCAP in the treatment of such stress-related brain or neuronal conditions and the use of TCAP in the preparation of a medicament for the treatment of such conditions.

### (e) Screening for Potential Modulators of TCAP Inhibition of Neruonal Cell Death.

In on embodiment, the invention provides a method for screening compounds that modulate TCAP inhibition of neuronal cell death, comprising, administering TCAP to neuronal cells under conditions that promote inhibition of neuronal cell death, such as pH induced cellular stress, in the presence of a potential TCAP modulator and monitoring the affects of said potential modulator on the viability of the neuronal cells. In one embodiment, this can be done in comparison to a control, such as the potential modulator with or without TCAP and/or with TCAP but no potential modulator. A deviation from the control would indicate the identification of a modulator. A modulator can enhance, decrease or normalize effects of TCAP. In another embodiment, the assay can be used to detect modulators of pH stress, such as neuronal cells. In one aspect of the invention the administration of TCAP can occur in a number of ways including, but not necessarily limited to: administering the TCAP in a suitable form of peptide to the cells, administering a substance that will enhance TCAP expression and/or availability of TCAP to the cell; administration of a nucleic acid encoding TCAP that will result in enhanced TCAP expression and/or availability to the cell.

### Pharmaceutical Compositions and Modes of Administration

TCAP, pharmaceutically acceptable salts or esters thereof or obvious chemical equivalents thereof can be administered by any means that produce contact of said active agent with the agent's sites of action in the body of a subject or patient to produce a therapeutic effect, in particular a beneficial effect, in particular a sustained beneficial effect. The active ingredients can be administered simultaneously or sequentially and in any order at different points in time to provide the desired beneficial effects. A compound and composition of the invention can be formulated for sustained release, for delivery locally or systemically. It lies with the capability of a skilled physician or veterinarian to select a form and route of administration that optimizes the effects of the compositions and treatments of the present invention to provide therapeutic effects, in particular beneficial effects, more particularly sustained beneficial effects.

In one embodiment, administration of TCAP includes any mode that produce contact of said active agent with the agent's sites of action in vitro or in the body of a subject or patient to produce the desired or therapeutic effect, as the ease may be. As such it includes administration of the peptide to the site of action—directly or through a mode of delivery (e.g. sustained release formulations, delivery vehicles that result in site directed delivery of the peptide to a particular cell or site in the body. It also includes administration of a substance that enhances TCAP expression and leads to delivery of TCAP to a desired cell or site in the body. This would include but is not limited to the use of a nucleotide encoding TCAP, e.g. via gene therapy or through a TCAP expression system in vitro or in vivo, as the case may be that results in enhanced expression of TCAP. It can also include administration of a substance to the cell or body that enhances TCAP levels at the desired site.

The above described substances including TCAP and nucleic acids encoding TCAP or other substances that enhance TCAP expression may be formulated into pharmaceutical compositions for administration to subjects in a biologically compatible form suitable for administration in vivo. By “biologically compatible form suitable for administration in vivo” is meant a form of the substance to be administered in which any toxic effects are outweighed by the therapeutic effects. The substances may be administered to living organisms including humans, and animals.

Thus in one embodiment, the invention provides the use of TCAP or modulator thereof in the preparation of a medicament for the inhibition of neuronal cell death and/or the treatment of related conditions. In one embodiment, a therapeutically effective amount of TCAP or a pharmaceutical composition as described herein is administered to a patient in need thereof. A patient in need thereof is any animal, in one embodiment a human, that may benefit from TCAP and its effect on inhibition of neuronal cell death.

An active substance may be administered in a convenient manner such as by injection (subcutaneous, intravenous, etc.), oral administration, inhalation, transdermal application, or rectal administration. Depending on the route of administration, the active substance may be coated in a material to protect the compound from the action of enzymes, acids and other natural conditions that may inactivate the compound. In one embodiment, TCAP is administered directly t or proximate to the desired site of action, by injection or by intavenous. If the active substance is a nucleic acid encoding, for example, a TCAP peptide it may be delivered using techniques known in the art.

The compositions described herein can be prepared by per se known methods for the preparation of pharmaceutical acceptable compositions which can be administered to subjects, such that an effective quantity of the active substance is combined in a mixture with a pharmaceutical acceptable vehicle or carrier. Suitable vehicles or carriers are described, for example, in Remington's Pharmaceutical Sciences (Remington's Pharmaceutical Sciences, Mack Publishing Company, Easton, Pa., USA 1985 or Remington's The Sciences and Practice of Pharmacey, 21^{st} Edition”, (University of the Sciences in Philadelphia, 2005) or Handbook of Pharmaceutical Additives (compiled by Michael and Irene Ash, Gower Publishing Limited, Aldershot, England (1995)). On this basis, the compositions include, albeit not exclusively, solutions of the substances in association with one or more pharmaceutical acceptable vehicles, carriers or diluents, and may be contained in buffered solutions with a suitable pH and/or be iso-osmotic with physiological fluids. In this regard, reference can be made to U.S. Pat. No. 5,843,456.

As will also be appreciated by those skilled, administration of substances described herein may be by an inactive viral carrier. In one embodiment TCAP can be administered in a vehicle comprising saline and acetic acid.

Further, in one embodiment, TCAP may be administered in a form that is conjugated to another peptide to facilitate delivery to a desired site, or in a vehicle, eg. a liposome or other vehicle or carrier for delivery. For instance, in one embodiment TCAP could be conjugated to a brain targeting vector, which is a peptide or peptidomimetic monoclonal antibody (MAb), that is transported into brain from blood via an endogenous blood brain barrier (BBB) transport system, which has shown to significantly reduce stroke volume (e.g. see Zhang et al. (2001)). Thus, in one embodiment, brain ischemia can be treated by neuropeptides, such as TCAP, with noninvasive intravenous administration. In one embodiment, the peptide is conjugated to a BBB drug targeting system such as transferrin, for example as described in Vuisser et al. (2004) or Kang et al. (1994). In another embodiment, TCAP does not require a transport mechanism to cross the blood brain barrier.

The present invention is described in the following Examples, which are set forth to aid in the understanding of the invention, and should not be construed to limit in any way the scope of the invention as defined in the claims which follow thereafter.

### EXAMPLES

### Example 1

### Peptide Synthesis

Mouse TCAP-1 (such as SEQ. ID. NO. 38) was prepared by solid phase synthesis as previously described (Qian et al., 2004). The peptide was solubilized in phosphate buffered saline (PBS) at a concentration of 2×10⁻⁷ M before being diluted in the appropriate medium.

### Example 2

### Cell Morphology Analysis

The effect of TCAP-1 on cell morphology was conducted using the N38 cells immortalized mouse hypothalamic cell line (Belsham et al, 2004). Cells were grown in six-well culture plate with 2 ml of Dulbeco's Modified Eagle Medium (DMEM) with high glucose, L-glutamate, 25 mM HEPES buffer, pyridoxine hydrochloride in the absence of sodium pyruvate, 5 ml penicillin with 10% fetal bovine serum (FBS) at pH 7.4 (all from Gibco-Invitrogen, Burlington, Canada).

At 24 and 48 hrs, the medium was replaced with medium buffered at pH 6.8, 7.4, 8.0 or 8.4. Half of the cell groups received (2×10⁻⁷ M) TCAP-1, whereas the other half received phosphate buffered saline (PBS) pH 7.4 containing 8 g NaCl, 0.2 g KCl, 1.4 g Na₂HPO₄, 0.2 g KH₂PO₄ in 800 mL ddH₂O. For all groups, 4 replicates were run. Digital pictures were taken at 24, 48 and 72 hrs using an Olympus IX&1 inverted microscope at a magnification of 200× and analyzed using LabWorks 4.0 Image Acquisition and Analysis software (UltraViolet Products Ltd).

### Results

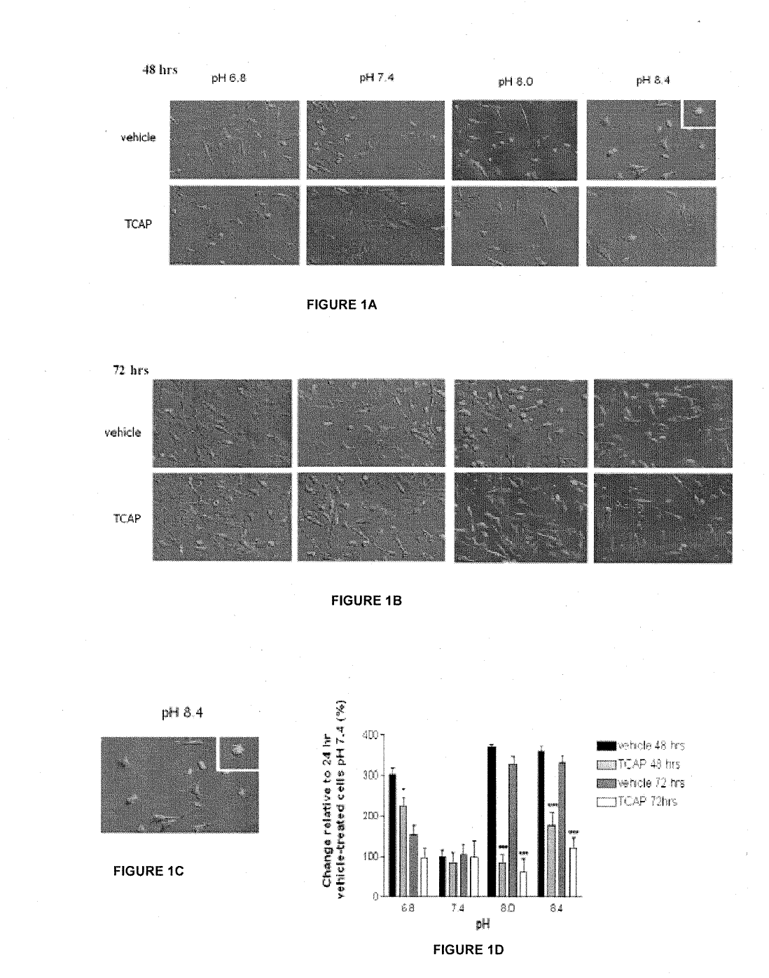

TCAP did not induce any observable morphological changes in the cells cultured at pH 7.4. However, there was significant increase in the number of small round cell types (necrotic cells) in the vehicle-treated cultures at pH 6.8 (p<0.05), 8.0 (p<0.001) and 8.4 (p<0.001) as compared to the TCAP-treated samples at 48 hrs (F=96.16). At 72 hrs, TCAP significantly decreased the number of rounded cells in pH 8.0 (p<0.001) and pH 8.4 (p<0.001) (F=51.13) relative to the vehicle-treated cells. (FIG. 1).

### Example 3

### Effect of TCAP on Cell Proliferation and Viability

The effect of TCAP-1 on cell proliferation at each pH was examined by direct counts using a hemocytometer and indirectly by assessing mitochondrial activity using a colorimetric MTT (3-[4,5-dimethylthiazol-2-yl]-2,5-diphenyl tetrazolium bromide) assay on cultured N38 cells. For hemocytometer counts, the cultures were incubated for 24 and 48 hrs. The cells were suspended using 1 ml of 0.25% Trypsin with EDTA (Gibco-Invitrogen, Burlington, Canada), centrifuged at 1600 RPM for 4 min, and resuspended with PBS. The cells in 50 μl aliquots were vortexed and counted on a hemocytometer.

The proportion of viable cells in the samples was determined by measuring Trypan Blue uptake. At 48 hrs, the cells from the four pH treatments were suspended using 1 ml of Trypsin EDTA, centrifuged at 1600 RPM for 4 min and resuspended in 1 ml of BSS (Hank's Balanced Salt Solution) (Sigma, St. Louis). An aliquot of 0.5 ml of 0.04% Trypan Blue solution was transferred to a 1.5 ml tube, 0.03 ml of BSS was added to 0.2 ml of the cell suspension; the samples were mixed thoroughly and the cell suspension-Trypan Blue mixture was allowed to stand for 10 minutes and then counted on a hemocytometer. Separate counts were kept for both viable and non viable cells.

A (3-4,5-dimethylthiazol-2-yl]-2,5-diphenyl tetrazolium bromide) MTT assay was conducted using the In Vitro Toxicology Assay Kit: MTT based (Sigma, St. Louis). The cells were cultured for the MTT assay at 24 and 48 hours and were incubated at 37° C. in 5% CO₂ for 3 hrs in the presence of MTT 200 μl/2 ml medium, The samples were mixed by shaking the plate horizontally for 30 min. The background absorbance of the multi-well plates was determined at 690 nm and subtracted from the 570 nm measurement.

### Results

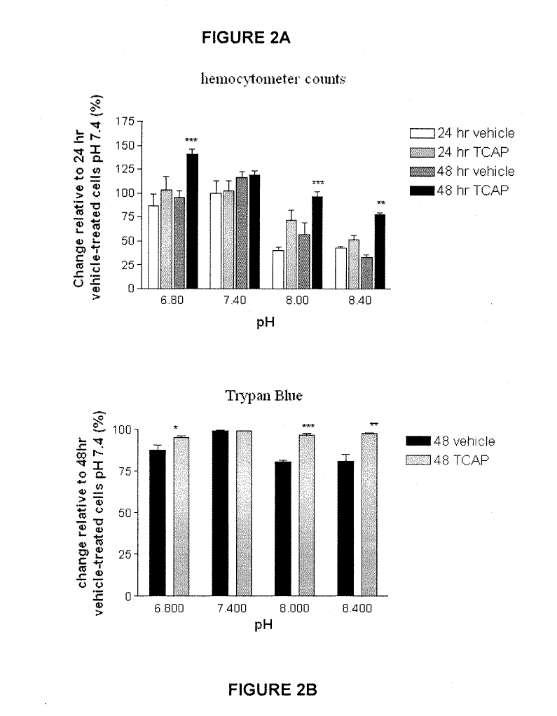

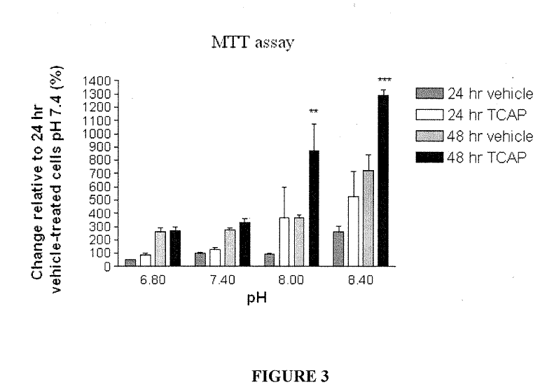

There were no significant differences in the total number of cells, as determined by direct hemocytometer counts, between the vehicle- and TCAP-treated cells at 24 hrs under any pH condition (FIG. 2a). There was a marked reduction in the number of total cells at pH 8.0 and 8.4 in the vehicle-treated cells at both 24 and 48 hrs. However, TCAP inhibited the decrease in total cell numbers relative to the vehicle-treated cells at pH 6.8 (P<0.001) 8.0 (P<0.001) and 8.4 (P<0.01) (F=38.10) after 48 hrs of incubation.

A Trypan Blue stain was conducted in order to estimate the proportion of viable cells in a population (FIG. 2b). TCAP-1 treatment caused a significant decrease in the number of cells that took up the trypan blue stain at 48 hrs in cells cultured at pH 6.8 (p<0.05), pH 8.0 (p<0.0001) and at pH 8.4 (p<0.001)(F=58.27) but not pH 7.4.

Although TCAP did not induce a significant effect on MTT activity at pH 7.4 or pH 6.8 there was a significant increase in optical density at 48 hrs in TCAP-1-treated samples cultured in pH 8.0 (p<0.01) and pH 8.4 (p<0.001) (F=21.19) (FIG. 3).

### Example 4

### Fluorescent Microscopy of Necrosis and Apoptosis Markers

N38 cells were cultured on poly-D-Lysine treated coverslips (VWR, Mississauga) in each of the four pH condition, and cells were washed twice with PBS, each fluorochrome was added to each well: 5 μl Fluorescein (FITC)-Annexin V in Tris EDTA buffer containing 0.1% BSA (Bovine serum albumin) and 0.1% NaN3, pH 7.5, 5 μl rhodamine EtD-III 200 μM in PBS and 5 μl 4′,6-Diamidino-2-phenylindole (DAPI) Hoechst 33342 5 μg/mL in PBS (Biotium, Inc. Hayward). The samples were incubated in the dark for 15 min, then washed before being placed on slides. The cells were viewed under a Leica DM4500 inverted fluorescent microscope and digitally analyzed using OpenLab software.

### Results

Annexin V labelled with fluorescein (FITC) was used to identify apoptotic cells in green. Ethidium homodimer III (EtD-III) is a positively charged nucleic acid probe, which is impermeable to live or apoptotic cells but stains necrotic cells with red fluorescence (rhodamine) and Hoechst 3342 (4′,6-Diamidino-2-phenylindole) (DAPI) emits bright blue fluorescence upon binding to DNA in living cells.

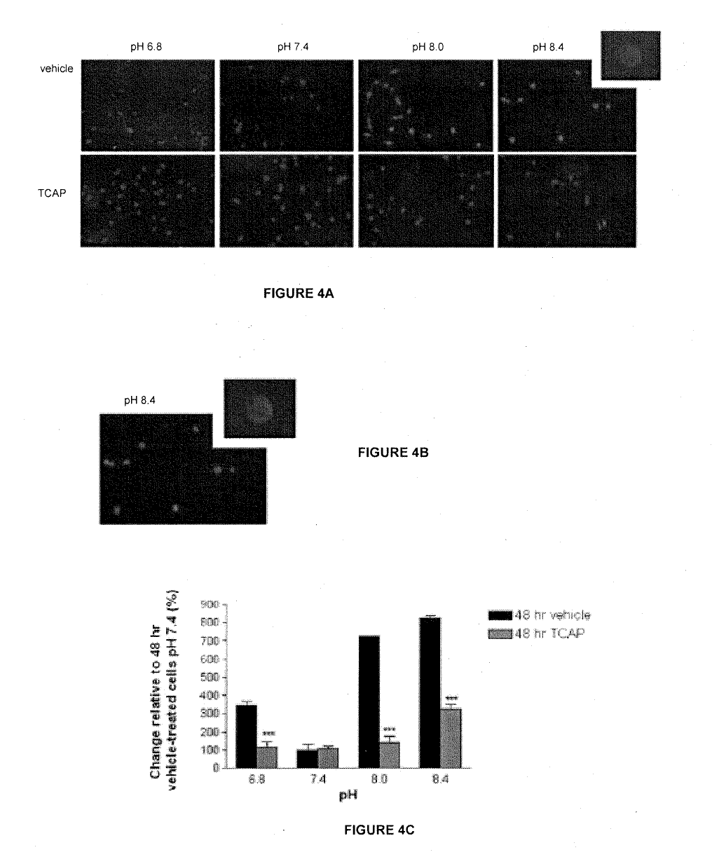

TCAP-1 decreased the number of rhodamine-fluorescing cells at pH 6.8 (p<0.001), 8.0 (p<0.001) and 8.4 (p<0.001) (F=348.2) but not in the pH 7.4 samples (FIG. 4). There were nominal amounts of FITC-labelled cells located intermittently throughout samples where only a total of 3 green cells were counted (see inset, FIG. 4).

### Summary of Examples 3 and 4

### Necrosis

Necrosis occurs when cells are exposed to extreme variance from physiological conditions such as hypothermia and hypoxia, which may result in damage to the plasma membrane (Majno and Jorris, 1995). Necrosis begins with an impairment of the cell's ability to maintain homeostasis, leading to an influx of water and extracellular ions. Intracellular organelles, most notably the mitochondria, and the entire cell swell and rupture (cell lysis) (Linnik et al, 1993). Due to the ultimate degeneration of the plasma membrane, the cytoplasmic contents including lysosomal enzymes are released into the extracellular fluid. Therefore, in vivo, necrotic cell death is often associated with extensive tissue damage resulting in an intense inflammatory response (Emery et al, 1993). Necrosis was determined as the form of cell death occurring based on expected morphological alterations affecting the plasma membrane including massive production of small surface evaginations (bubbles) caused by the cells inability to control water influx through the plasma membrane (Rello et al., 2005). The Trypan Blue Stain (Example 3) is based on an acid dye that contains two azo chromophores. The reactivity of this dye is dependent on the negatively charged chromophore binding to cytoplasmic material when the membrane is damaged. Staining facilitates the visualization of cell morphology since it is only the dead cells that take up the dye, thus identifying cells that are necrotic or are in the very late stages of apoptosis. The fluorescent microscopy study (Example 4) also solidifies this assumption as TCAP decreases the number of necrotic cells and not apoptotic cells. These findings are significant as necrosis plays an integral role in neurodegenerative diseases.

### Example 5

### Apoptosis (Caspase and PARP) Markers

Apoptosis, otherwise known as “programmed cell death” is a necessary event of normal development. The apoptotic pathway is mediated by a family of death proteins, caspases. These signaling proteins are proteolytic enzymes that when inactive, lay dormant as zymogens, until they are activated by various triggers (Hengartner, 2000). Upon activation of caspase 3 certain nuclear proteins are cleaved such as poly ADP-ribose polymerase (PARP). PARP, a 116 kDa nuclear polymerase, is involved in DNA repair usually in response to environmental stress (Hengartner, 2000; Willie, 1980; Kerr, 1972). The protein can be cleaved by many interleukin-converting enzyme-like (ICE-like) proteases (Willie, 1980; Liu, 1997). (PARP) was one of the first proteins reported to be cleaved during apoptosis, and is a target of the Yama/CPP32 protease, caspase-3 (Kaufmann, 1989; Kaufman et al, 1993). Cleavage products occurring due to apoptosis result in western blot bands at 89 KDa. The following experiments were conducted to determine whether TCAP works through the apoptotic pathway.

### (a) Calorimetric Caspase Assays

Caspase 8 and 3 colorimetric assays were performed on the N38 cells at all pH conditions. The assay was based on the detection of the chromophore pNA after cleavage from the labeled substrate IETD-pNA and DEVD-pNA for caspase 8 and 3, respectively. Comparison of the pNA absorbance from the suspected apoptotic sample was compared to the uninduced neutral pH sample. Caspase 8 and 3 were analysed using the Caspase-3 Colorimetric Activity Assay (Chemicon, Temecula USA) and Caspase-8 Colorimetric Activity Assay (Chemicon, Temecula USA). The cells from each pH treatment described previously at 24 and 48 hrs were removed using a cell scraper and centrifuged at 1500 rpm for 10 minutes. The cells were resuspended in 350 μl of chilled cell lysis buffer containing 500 μl PBS, 5 μl 1% Triton×100 (Sigma, St. Louis), 25 μl proteinase inhibitor cocktail set III (VWR, Mississauga), 0.5 μl 1 M dithiothreitol (DTT) (Sigma, St. Louis) and 2.5 μl phenylmethylsulphonylfluoride (PMSF) diluted in 1 mL of methanol (EM Science, Gibbstown), then incubated on ice for 10 min and centrifuged for 5 minutes at 10,000 rpm, The supernatant, consisting of cytosolic extracts, was transferred to a new tube and a bicinchoninic acid (BCA) protein assay (Pierce, Rockford) was conducted to determine total protein concentration. The absorbance of each sample was measured on a SPECTRAmax Microplate spectrophotometer at 405 nm after an incubation period of 2 hours at 37° C. Changes in caspase 3 activity were determined by comparing the absorbance reading from the induced sample with the level of the uninduced control. Background readings from the buffer were subtracted from the reading of both the induced (pH 6.8, 8.0, 8.4) and uninduced (pH 7.4) samples before calculating changes in caspase 3 activity. The same was done for the detection of caspase 8. As a control, N38 cells were cultured with pH 7.4 DMEM and incubated for 4 days, apoptosis was then induced using 10 μM/ml etoposide and lysed according to the above protocol and used a control for all subsequent caspase 3 detection. All assays were performed with 4 replications.

### (b) Caspase 3 and Poly(ADP-Ribose)Polymerase (PARP) Cleavage by Immunoblot

Detection of caspase 3 cleavage was determined at 48 hrs. The samples at each pH and control (see above) were lysed using total protein isolation lysis buffer (described above). An aliquot of 25 μl of each sample was combined with 25 μl of 2×20% sodium dodecyl sulphate (SDS) sample buffer and loaded onto a 4-10% HCL-Tris pre cast polyacrylamide gel (BioRad, Mississauga). The gel was run at 200 v for 35 min and proteins were electrotransfered to a Hybond-C nitrocellulose membrane (Amersham, Baie d'Urfé) for 75 min at 100 v. After transfer, the membrane was washed with 10 ml of PBS with 0.05% Tween 20 (PBST) for 5 min at room temperature (RT) and the membrane was incubated in 10 ml of PBST-milk for one hour at RT followed by 3 times for 5 min washes with 10 ml of PBST. The membrane was then incubated with cleaved caspase 3 primary antibody (Cell Signaling Technology, Beverly) at a titre of 1:500 in 6 ml of PBST-milk with gentle agitation overnight at 4° C. The membranes were washed 3 times for 5 min with 10 ml of PBST followed by membrane incubation with anti-rabbit horseradish peroxidase (HRP)-conjugated secondary antibody (Amersham, Baie d'Urfé) at 1:3000 in 6 ml of PBST-milk with gentle agitation for 1 hr at RT. The membranes were then washed 3 times for 5 min with 10 ml of PBST then exposed to Kodak X-OMAT Blue scientific imaging film (Perkin Elmer Canada Inc, Vaudreuil-Dorion) for 30 min.

Using the same protocol, changes in PARP expression were determined at 48 hrs. The membrane was incubated with PARP primary antibody (Cell Signaling Technology, Beverly) at a titre of 1:100. The membranes were washed 3 times for 5 min with 10 ml of PBST followed by membrane incubation with anti-rabbit horseradish peroxidase (HRP)-conjugated secondary antibody (Amersham, Baie d'Urfé) at 1:3000 in 6 ml of PBST-milk with gentle agitation for 1 hr at RT. The membranes were then washed 3 times for 5 min with 10 ml of PBST then exposed to Kodak X-OMAT Blue scientific imaging film (Perkin Elmer Canada Inc, Vaudreuil-Dorion) for 30 min. Total optical density of the blots, were quantified using LabWorks 4.0 Image Acquisition and Analysis Software from Ultra-Violet Products Ltd. (UVP).

### Results

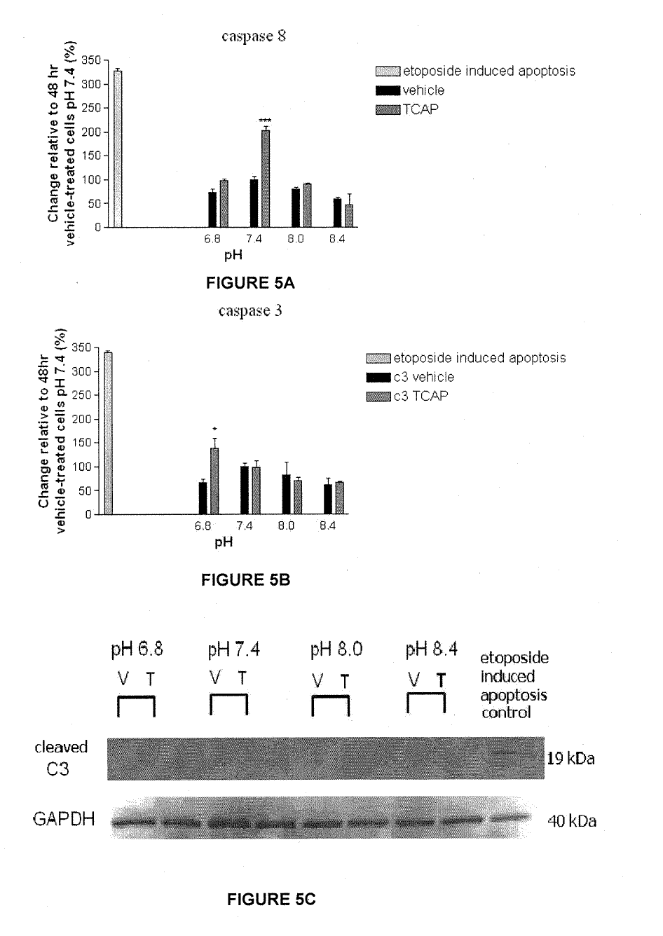

Etoposide (cone) was used to determine the amount of caspase 8 (FIG. 5a) and 3 (FIG. 5b) activation under apoptotic conditions. Etoposide induced a greater than 3-fold increase in caspase 8 and 3.5-fold increase in caspase-3 relative to the vehicle-treated cells at pH 7.4. Although TCAP-1 increased caspase 8 activity in pH 7.4 samples (P<0.001)(F=20.80) and increased caspase 3 activity in pH 6.8 samples (P<0.05) (F=2.117), the relative level of caspase activity was about 70% and 40% of the etoposide-induced increase for caspase 8 and 3 respectively. There were no significant differences in caspase 8 and 3 activity between the TCAP-1- and vehicle-treated cells at pH 8.0 and 8.4. As a further determination of caspase 3 activity, four replicates of western blots were conducted on pH treated. N38 cells at the 48 hr mark in order to detect the cleaved and activated caspase 3 (17/19 kDa) (FIG. 5c). The caspase 3 cleavage product was clearly visible in the protein extracts of the etoposide-treated cell but could not be observed in any of the TCAP-1 or vehicle-treated cells at any of the pH conditions.

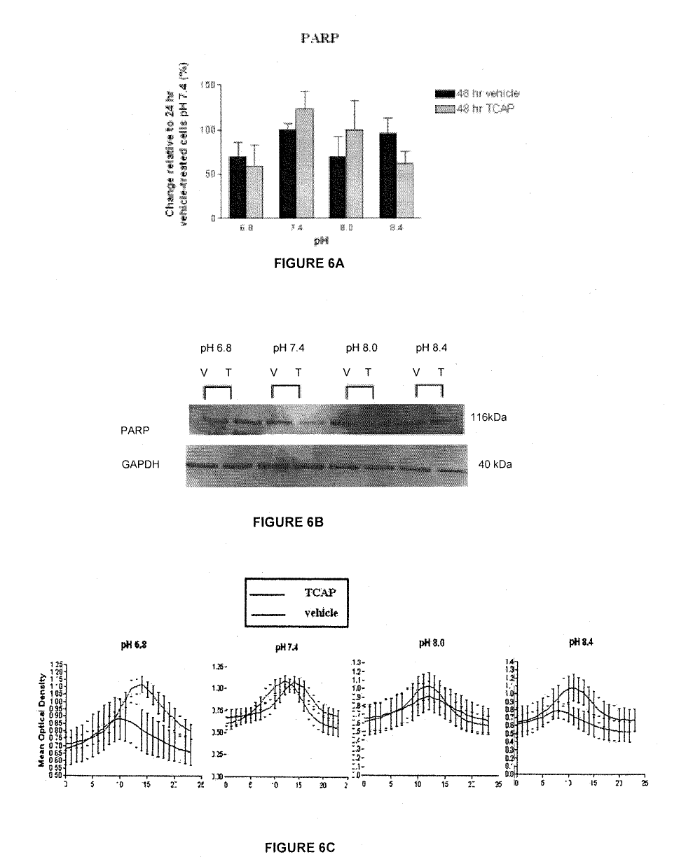

Four replicates of western blots were conducted on pH treated N38 cells at the 48 hour mark in order to detect endogenous levels of full length PARP, as well as the large fragment (89 kDa) and small fragment (24 kDa) of PARP resulting from caspase cleavage. The western blot revealed endogenous PARP at all pH treatments as well as vehicles samples and based on a two way ANOVA using Bonferroni's Post Test, there were no significant differences between vehicle and TCAP treated samples (FIG. 6).

Based on the studies conducted and described in Example 5, TCAP is not protecting neuronal cells by inhibiting the apoptotic pathway.

### Example 6

### Kinase B/Akt Cell Survival Pathway

Protein kinase B or Akt (PKB/Akt) is a serine/threonine kinase, which functions to promote cell survival by inhibiting apoptosis by means of its ability to phosphorylate and inactivate several targets including BAD and forkhead transcription factors (Crowder, 1998). AKT, also referred to as PKB or Rae, plays a critical role in controlling the balance between cell survival and cell death in neurons (Dudek, 1997). The present example was conducted to determine whether TCP acts through this particular survival pathway.

Western blots using Akt and phosphorylated Akt (P-Akt) primary antibodies were conducted on all conditions of the cultured N38 cells to determine whether TCAP was preventing cell death by phosphorylation. The same western blot procedure outlined above was repeated with an Akt primary antibody (Cell Signalling, Beverly) at a titer of 1:500, followed by membrane incubation with anti-rabbit HRP-conjugated secondary antibody (Amersham, Baie d'Urfé) at 1:3000, followed by exposure on Kodak X-OMAT Blue film (Perkin Elmer Canada Inc, Vaudreuil-Dorion) for 30 min. Phospho Akt expression at 48 hrs was determined using the method described above with a PAkt primary antibody (Cell Signalling 9271) at 1:1000 followed by membrane incubation with anti-rabbit HRP-conjugated secondary antibody (Amersham, Baie d'Urfé) at 1:2000 followed by exposure on Kodak X-OMAT Blue film (Perkin Elmer Canada Inc, Vaudreuil-Dorion) overnight. Cultured N38 cells were serum-starved for 48 hours in order to induce phosphorylation and following the same protocol above were loaded as a control. Total optical density of the blots, were quantified using LabWorks 4.0 Image Acquisition and Analysis Software from Ultra-Violet Products Ltd. (UVP).

### Results

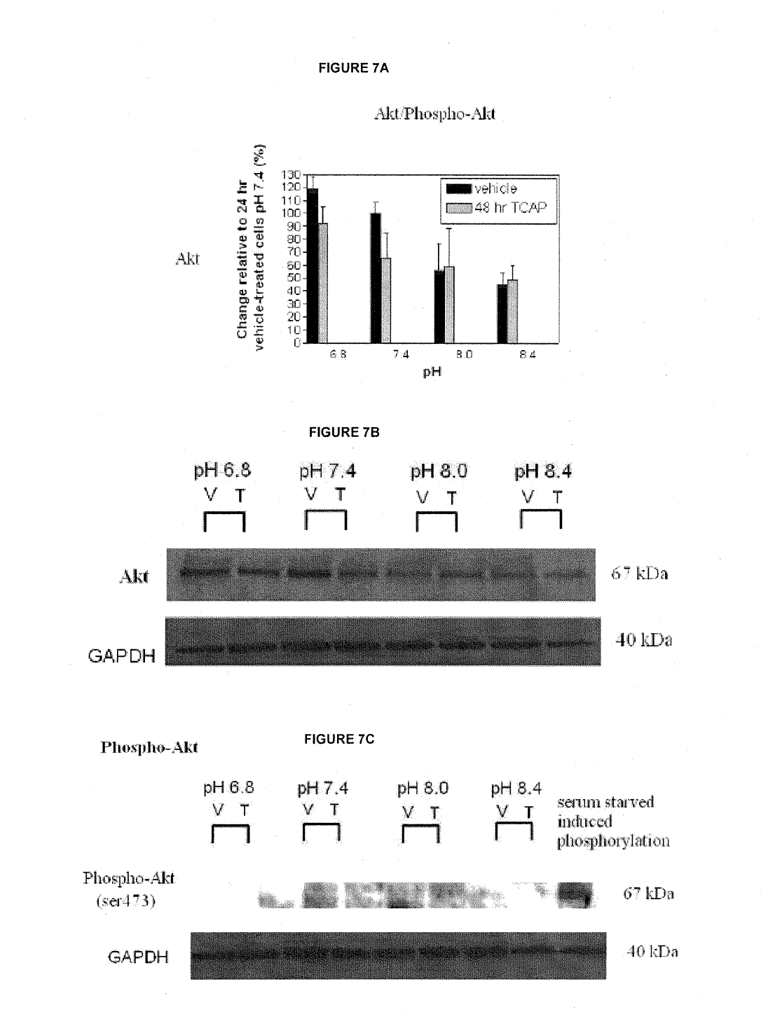

Western blots were conducted using an Akt antibody, which detected total levels of endogenous Akt. The blot revealed endogenous Akt in all treatments as well as the vehicle, however according to a two way ANOVA using Bonferroni's Post Test, there appears to be no difference in endogenous Akt between vehicle and TCAP treated samples. Total optical density of the blots were quantified using LabWorks 4.0 Image Acquisition and Analysis Software from Ultra-Violet Products Ltd. (UVP) (FIG. 7b).

Western blots were conducted using a Phospho-Akt antibody, which detected total levels of endogenous Akt1 only when phosphorylated at serine 473. The blot revealed no bands in any samples, thus phosphylation of Akt is not occurring. Phosphorylation of cells was induced by serum starvation and loaded as a control, the blot revealed a band, however no other bands were detected (FIG. 7c).

### Example 7

### The Effect of TCAP On Cell Cycling: Bromodeoxyuridine (BrdU)

### Incorporation Assay

The evaluation of cell cycle progression is important when assessing the viability of a cell population. The cell cycle is a sequence of stages that a cell passes through between one division and the next. The cell cycle oscillates between mitosis and the interphase, which is divided into G, S, and G 2. In the G phase there is a high rate of biosynthesis and growth; in the S phase there is the doubling of the DNA content as a consequence of chromosome replication; in the G 2 phase the final preparations for cell division (cytokinesis) are made (Raza, 1985). In order to determine whether TCAP was increasing cell cycle efficiency, a bromodeoxyuridine (BrdU) non-isotopic enzyme immunoassay was conducted (Calbiochem, Canada). BrdU incorporation into newly synthesized DNA of actively proliferating cells enables one to quantify cell cycle progression and the population of cells entering the S phase (Gratzner, 1982; Raza, 1985).

N38 cells were grown in a 96-well culture plate using 100 μl at an initial density of 2×10⁵ cells/ml. Controls consisted of a blank, one well containing only DMEM with no cells and background, and one well with cells but with no BrdU label added. A working stock of BrdU was prepared by diluting the BrdU label 1:2000 into fresh DMEM, 200 of the working stock was added to each well to be labelled, the BrdU was allowed to incubate with the cells for 2 hrs at 37° C. The contents of the wells were then removed and 2000 of the enclosed Fixative/Dentauring solution was added to each well and incubated for 30 min at Room Temperature (RT). The contents of the wells were removed and Anti-BrdU Antibody (1:100) was added to each well and incubated for 1 hr at RT. Wells were washed 3 times with wash buffer, the plate was then gently blotted on paper towel. The conjugate was prepared by diluting the reconstituted in (1×PBS) peroxidase goat anti-Mouse IgG HRP conjugate in the enclosed conjugate diluent and loaded onto a syringe filter through 0.2 micron filter and a 100 μl aliquot of this solution was transferred to each well and incubated for 30 min at RT. The wells were washed with wash buffer, the entire plate was then flooded with double deonized water and the contents of the wells were removed. An aliquot of 100 μl of BrdU substrate solution was added to each well, the plate was then incubated in the dark at RT for 15 min. 100 μl of stop solution containing 2.5N sulphuric acid was added to each well in the same order as the previously added substrate solution. Absorbance was measured on a SPECTRAmax Microplate spectrophotometer at dual wavelengths at 450-540 nm.

### Results

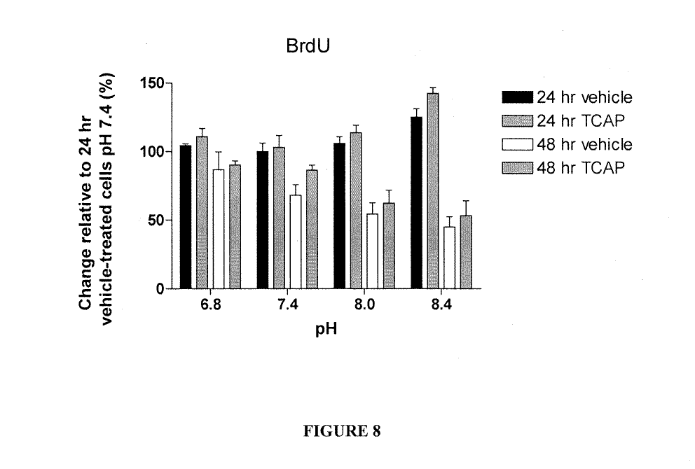

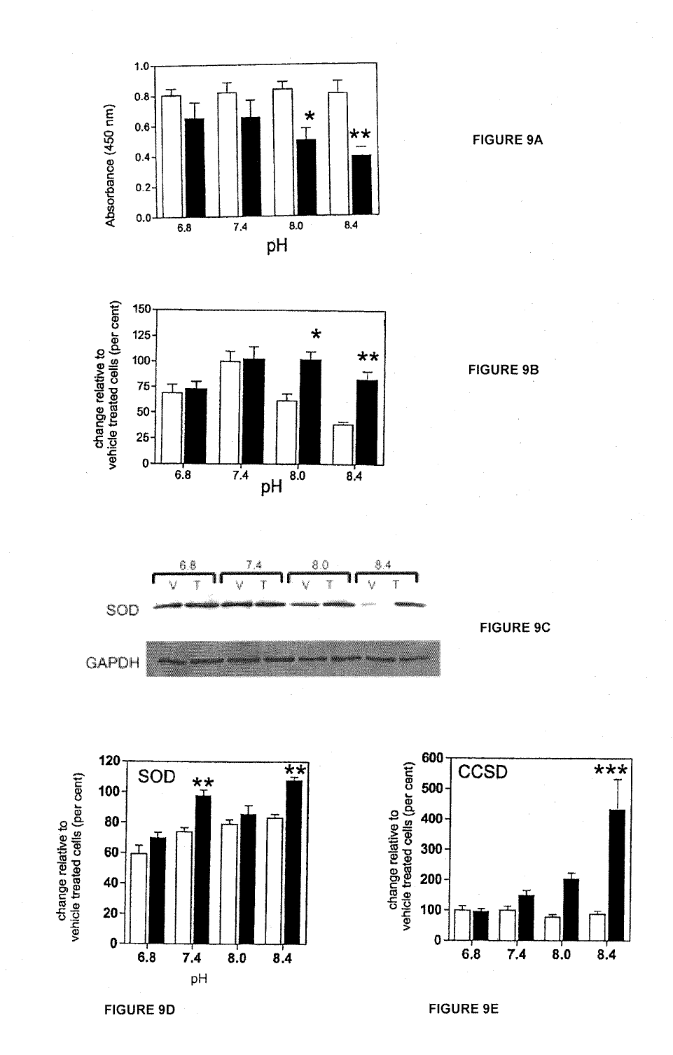

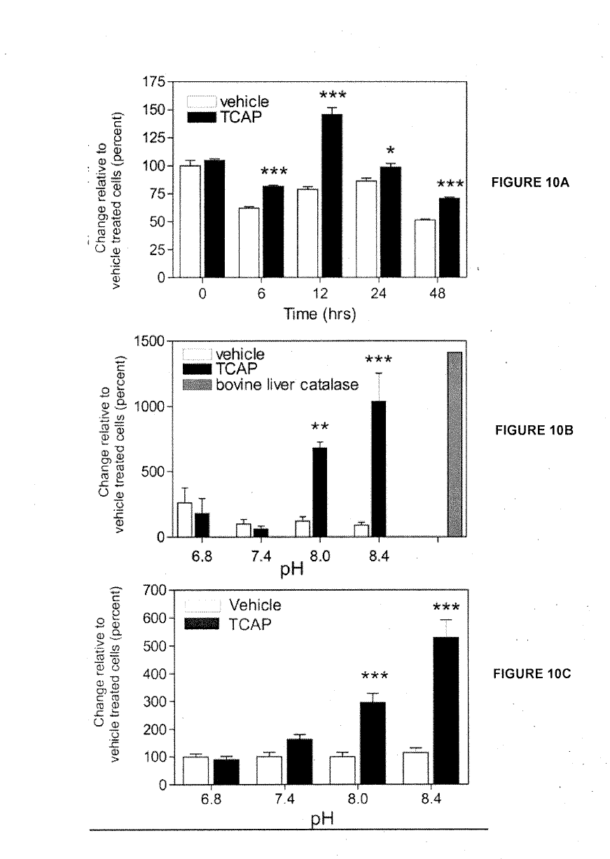

Based on a two way ANOVA using Bonferroni's Post Test there were no significant results at 24 or 48 hrs (FIG. 8).

This investigation indicates that synthetic TCAP-1 has a neuroprotective effect on immortalized hypothalamic mouse cells. The data described in this study suggest a significant neuroprotective role for TCAP during times of pH induced cellular stress. Several lines of evidence point to this. Based on haemocytometer counts and an MTT assay conducted on pH stressed N38 cell samples, TCAP has a positive affect on cell viability during pH induced cellular stress, suggesting that TCAP could be inhibiting cells from undergoing apoptosis, acting through a cell survival pathway or rescuing cells from necrosis. The Examples herein indicate that this neuroprotective effect occurs by the inhibition of mechanisms regulating necrosis and to a lesser extent by regulating apoptotic, survival, or cell cycle pathways.

### Example 8

### Superoxide Dismutase-Catalase Data

### Superoxide Dismutase Detection and Measurement

Examination of the superoxide dismutase-associated system was investigated as a possible mechanism for necrosis. The presence of the superoxide radical was measured indirectly by the conversion of a soluble tetrazolium salt in cells after 48 hours (FIG. 9A). The TCAP-1 treated cells showed a 40% (p<0.05) and 60% (p<0.01) decrease in the absorbance of the substrate, which is proportional to superoxide radical activity, at pHs 8.0 and 8.4, respectively. However, because this method shows only the indirect presence of the superoxide radical, and by inference, the presence of superoxide dismutase, we also examined the presence of this enzyme protein directly by western blot (FIG. 9B,C). Relative to the vehicle-treated cells at pH 7.4, superoxide dismutase levels in the vehicle-treated cells showed a significant (p<0.05) decrease as a function of pH, as determined by a one-way ANOVA. There were no significant differences in the expression of the superoxide dismutase protein at pHs 6.8 and 7.4. In contrast, at pH 8.0 and 8.4, TCAP-1 significantly (p<0.05 and p<0.01, respectively) reduced the pH-induced decline in superoxide dismutase levels. The superoxide dismutase expression levels at pH 8.0 and 8.4 were not significantly different than that of the vehicle-treated cells at pH 7.4.

Superoxide dismustase gene expression as measured by real-time PCR indicated a significant (p<0.01) increase over the vehicle treated cells at pH 7.4 and 8.4 (FIG. 9D). A greater effect on gene expression was noted in superoxide copper chaperone (CCSD) expression where CCSD expression levels in the TCAP-1 treated cells at pH 8.4 was increased almost 4.5 fold over the vehicle treated cells (FIG. 9E).

H₂O₂ Toxicity and Catalase Activity

TCAP-1 showed a significant increase in MIT activity relative to the vehicle-treated at 6 to 48 hours in cells treated with 50 μM H₂O₂ (FIG. 10A). The results indicate that TCAP-1 significantly increased mitochondrial activity at 6, 12 and 48 hours (p<0.001) (F=168.2) as compared with the vehicle-treated cells. There was also a less significant effect at 24 hours (p<0.05) and no effect at 0 hours.

A catalase assay was performed on the pH treated cells in order to determine whether TCAP-1 was conferring survivability to the cells via upregulation of catalase and thus increasing H₂O₂ breakdown into H₂O and O₂ (FIG. 10B). The results indicate that TCAP-1 significantly increased catalase levels at pH 8.4 (p<0.001)(F=24.42) as compared to the vehicle treated cells according to a two-way ANOVA with a Bonferroni's post hoc test. There was also a significant TCAP-1 effect at pH 8.0 (p<0.01) but no significant effects at either pH. 6.8 or pH 7.4 compared to the vehicle treated cells. Bovine liver was also assayed as a positive control. Catalase gene expression, as determined by real-time PCR indicated that TCAP induced mRNA levels by 3 fold (p<0001) and 5 fold (p<0.001) at pHs 8.0 and 8.4, respectively.

While the foregoing invention has been described in some detail for purposes of clarity and understanding, it will be appreciated by one skilled in the art, from a reading of the disclosure, that various changes in form and detail can be made without departing from the true scope of the invention in the appended claims.

All publications, patents, and patent applications are herein incorporated by reference in their entirety to the same extent as if each individual publication, patent or patent application was specifically and individually indicated to be incorporated by reference in its entirety.

### REFERENCES

1. Akbar M, Calderon F, Wen Z, Kim H Y. Docosahexaenoic acid: A positive modulator of Akt signaling in neuronal survival. Proc Natl Acad Sci USA. 2005 Jul. 22; [Epub ahead of print]
2. Arends, M. and Willie Aft Apoptosis: mechanisms and role in pathology. Int Rev. Exp. Pathol. 32 (1991):223-254.
3. Baumgartner, S., Martin, D., Hagios, C. & R. Chiquet-Ehrismann, Tenm, a Drosophila gene related to tenascin, is a new pair rule gene. EMBO J. 13 (1994) 3728-3740.
4. Belsham, D., Cai, F., Cui, H., Smukler, S., Salapatek, A., Shkreta, L. Generation of a Phenotypic Array of Hypothalamic Neuronal Cell Models to Study Complex Neuroendocrine Disorders. Endocrinology 145 (2004): 393-400.
5. Brenneman, D., J. M. Hill, G. W. Glazner, I. Gozes and T. W. Phillips. Interleukin-1 alpha and vasoactive intestinal peptide: enigmatic regulation of neuronal survival. Int. J. Dev. Neurosci. 13 (1995):187-210.
6. Buja, L M., Eigenbrodt M L and Eigenbrodt E H. Apoptosis and necrosis: basic types and mechanisms of cell death. Arch. Pathol. Lab. Med. 117 (1993):1208-1214.
7. Casiano C, Ochs R, Tan, E Distinct cleavage products of nuclear proteins in apoptosis and necrosis revealed by autoantibody probes. Cell Death and Differentiation. 5 (1998): 183-190.
8. Columbano A. Cell death: current difficulties in discriminating apoptosis from necrosis in the context of pathological processes in vivo. J. Cell. Biochem. 58 (1995):181-190.
9. Crowder R J, Freeman R S. Phosphatidylinositol 3-kinase and Akt protein kinase are necessary and sufficient for the survival of nerve growth factor-dependent sympathetic neurons. J Neurosci 18 (1998): 2933-2943.
10. Dudek H, Datta, S., Franke, T., Birnbaum, M., Yao R, Cooper, G., Segal, R., Kaplan, D., Greenberg, M. Regulation of neuronal survival by the serine-threonine protein kinase Akt. Science. 275 (1997): 661-5
11. Emery E., Aldanap, P., Bunge, M B. Apoptosis after traumatic human spinal cord injury. J. Neurosurg 89 (1998)911-20.
12. Gennari, J. Goldstein, M & W. Schwartz, The nature of the renal adaptation to chronic hypocapnia. J Clin Invest. 1972 July; 51(7): 1722-1730.
13. Giffard R G, Weiss J H, Choi D W. Extracellular alkalinity exacerbates injury of cultured cortical neurons. Strok. 23 (1992): 1817-1821.
14. Glazer, R., and 1. Gozes. Diurnal oscillation in vasoactive intestinal peptide gene expression independent of environmental light entraining. Brain Res. 644 (1994):164-168.
15. Gozes, I. and D. E. Brenneman. Activity-dependent neurotrophic factor (ADNF): An extracellular neuroprotective chaperonin? J. Molec. Neurosci. 7 (1996):235-244.
16. Gratzner, H. G. Monoclonal antibody to 5-bromo- and 5-iododeoxyuridine: A new reagent for detection of DNA replication. 218 (1982):474-5.
17. Hengartner M O. The biochemistry of apoptosis. Nature. (2000);407(6805):770-6.
18. Kang Y. S., Bickel, U., Pardridge, W. M. 1994 Pharmacokinetics and saturable blood brain barrier transport of biotin bound to a conjugate of avidin and a monoclonal antibody to the transferring receptor Drug Metab. Dispos 22, 99-105.
19. Kaufmann, S H. Induction of endonucleolytic DNA cleavage in human acute myelogenous leukemia cells by etoposide, camptothecin and other cytotoxic anticancer drugs: a cautionary note. Cancer Res. 49 (1989):5870-5878,
20. Kaufmann, S H., Desnoyers, S., Ottaviano, Y., Davidson N E and Poirier, G G. Specific proteolytic cleavage of poly(ADP-ribose) polymerase: an early marker of chemotherapy-induced apoptosis. Cancer Res. 53 (1993):3976-3985.
21. Khaled, A., Bulavin, D., Kittipatarin, C., Li, W., Alvarez, M., Kim, K., Young, H., Fornace, A., Durum, S. Cytokine-driven cell cycling is mediated through Cdc25A. JCB 169 (1999): 755-763.
22. Levine, A., Bashan-Ahrend, O., Budai-Hadrian, D., Gartenberg, S., Menasherow, R., Wides, R. Odd Oz: a novel Drosophila pair rule gene. Cell. (1994): 587-598.
23. Levine, R., Helpern, J., Welch, A., Vande Linde, A., Sawaya, K., Brown, E., Ramadan, N., Deveshwar, R., Ordidge, R. Human focal cerebral ischemia: evaluation of brain pH and energy metabolism with P-31 NMR spectroscopy. Radiology 185. (1992): 537-544.
24. Linnik, M D., Zobrist R H., Hatfield M D. Evidence supporting a role for programmed cell death in focal cerebral ischemia in rats. Stroke 24 (1993)2002-2009.
25. Liu, X., Zhou, H., Slaughter, C., Wang X. DEF, a heterodimeric protein that functions downstream of caspase-3 to trigger DNA fragmentation during apoptosis. Cell 89 (1997): 175-84.
26. Mabe H, Blomqvist P, Siesjo B K. Intracellular pH in the brain following transient ischaemia. J Cereb Blood Flow Metab. 3 (1993): 109-114.
27. Majno G and Jorris L. Apoptosis, oncosis and necrosis: an overview of cell death. Amer J. Pathol. 146 (1995):3-15.
28. Minet, A. and Chiquet-Ehrismann, R. Phylogenetic analysis of teneurin genes and comparison to the rearrangement hot spot elements of E. Coli. Gene. 257 (2000): 87-89.
29. Minet, A., Rubin, B., Tucker, R., Baumgartner, S., Chiquet-Ehrismann, R. Teneurin-1, a vertebrate homologue of the Drosophilapair-rule gene Ten-m, is a neuronal protein with a novel type of heparin-binding domain. Journal of Cell Science 112 (1999): 2019-2032.
30. Moon R B, Richards J H. Determination of intracellular pH by 31P MR. J Biol Chem 1973; 248: 7276-7278.
31. Oohashi, T., Zhou, K., Feng, B., Richter, M., Morgelin, M., Perez, W., Su, R., Chiquet-Ehrismann, R., Rauch, U., Fassler, R. Mouse ten-m/Odz is a new family of dimeric type II transmembrane proteins expressed in many tissues. J. Cell Biol. 145 (1999):563-577.
32. Ostermann M E, Girgis-Hanna Y, Nelson S R, Eastwood J B, Metabolic alkalosis in patients with renal failure, Nephrol Dial Transplant. 18 (2003):2442-8.
33. Pawloski, J., Kraft, A. Bax induced apoptotic cell death. PNAS. 97 (2000):529-531.
34. Potapenko E, Kostyuk E, Voitenko N, Kostyuk P. Alkalinization-induced changes in intracellular calcium in rat spinal cord neurons. Neurochem Res. 2004 September; 29(9):1659-65.
35. Priest, J. M, Fischbeck, K. H., Nouri, N & B. J., Keats, A locus for axonal motorsensory neuropathy with deafness and mental retardation maps to Xq24-q26. Genomics 29 (1995) 409-412.
36. Qian X, Barsyte-Lovejoy D, Wang L, Chewpoy B, Gautam N, Al Chawaf A, D. A Lovejoy, Cloning and characterization of teneurin C-terminus associated peptide TCAP)-3 from the hypothalamus of an adult rainbow trout (Oncorhynchus mykiss). Gen Comp Endocrinol. 137 (2004):205-16.
37. Raza, A., Ukar, K., Preisler, H D. Double labeling and in vitro versus in vivo incorporation of bromodeoxyuridine in patients with acute nonlymphocytic leukemia. Cytometry 6 (1985):633-40.
38. Rello, S., Stockert, J., Moreno, V., Gamez, A., Pacheco, M., Juarranz, A., Canete, M., Villanueva, A. Morphological criteria to distinguish cell death induced by apoptotic and necrotic treatments. Apoptosis. 10 (2005):201-208.
39. Robertson N J, Cowan F M, Cox I J, Edwards A D Brain alkaline intracellular pH after neonatal encephalopathy. Ann. Neurol. 52 (2002):732-742.
40. Rosser B G and Gores G J. Liver cell necrosis: cellular mechanisms and clinical implications. Gastroenterology. 108 (1995):252-275.
41. Rubin, B., Tucker, P., Martin, D., Chiquet-Ehrismann, R. Teneurins: a novel family of neuronal cell surface proteins in vertebrates, homologous to the Drosophila pair-rule gene product, Ten-m, Dev Biol. 216 (1999): 195-209.
42. Sapolsky, R. M. (1992) Stress, the aging brain and the mechanisms of neuronal death. MIT Press. Cambridge Mass. 428 pages.
43. Stout, A., Raphael, H., Kanterewicz, B., Klann, E., Reynolds, I. Glutamate-induced neuron death requires mitochondrial calcium uptake. Nature Neuroscience. 1 (1998): 366-373.
44, Thornton J S, Ordidge R J, Penrice J, Cady E B, Amess P N, Punwani S, Clemence M, Wyatt J S, Temporal and anatomical variations of brain water apparent diffusion coefficient in perinatal cerebral hypoxic-ischemic injury: relationships to cerebral energy metabolism. Magri Reson. Med. 39 (1998):920-927.
45. Thompson, C B. Apoptosis in the pathogenesis and treatment of disease. Science 267 (1995):1465-1462.
46. Traynelis S, Cull-Candy S. Proton inhibition of NMDA receptors in cerebellar neurons. Nature. 356 (1990): 347-349.
47, Vornov J J, Thomas A G, Jo D. Protective effects of extracellular acidosis and blockade of sodium/hydrogen ion exchange during recovery from metabolic inhibition in neuronal tissue culture. J. Neurochem. 67 (1996): 2379-2389.
48. Vuisser, C. C., Stevanovic, S., Voorwindern, L., Gaillard, P. J., Cormmelin, D. J., Danhof, M., DeBoer A G 2004 Validation of the transferring receptor for drug targeting to brain capillary endothelial cells in vitro. J. Drug Target, 12, 145-150.
49. Wang, L. S. Rotzinger, A. AlChawaf, R. B. Chewpoy D. Barsyte-Lovejoy, X. Qian, C. F. Elias, N. C. Wang, J. C. Bittencourt, A. De Cristefaro, D. Belsham, F. Vaccarino, D. A. Lovejoy, Teneurin proteins possess a carboxy terminal (CRF)-like sequence that modulates emotionality and neuronal growth, Submitted to Molecular Brain Research (2004).
50. Watson, A J M. Necrosis and apoptosis in the gastrointestinal tract. Gut 37 (1995):165-167.
51. Willie, A H. Glucocorticoid-induced thymocyte apoptosis is associated with endogenous endonuclease activation. Nature 284 (1980):555-6.
52. Willie, A H., Kerr, J F R and Currie A C. Cell death: the significance of apoptosis. Int. Rev. Cytol. 68 (1980):251-305.
53. Xu L, Glassford A J M, Giaccia A J, Giffard R G. Acidosis reduces neuronal apoptosis. Neuroreport. 9 (1998): 875-879.
54. Zhang Y, Pardridge W M. Conjugation of brain-derived neurotrophic factor to a blood-brain barrier drug targeting system enables neuroprotection in regional brain ischemia following intravenous injection of the neurotrophin. Brain Res 889 (2001): 49-56.

What is claimed is:

1. A method of inhibiting neuronal cell death comprising administering to the cell an effective amount of Teneurin C-Terminal Associated Peptides (TCAP), a pharmaceutical acceptable salt or ester thereof or obvious chemical equivalent thereof.

2. The method of claim 1, wherein inhibiting neuronal cell death comprises protecting neuronal cells from cell death under conditions where cell death may occur.

3. The method of claim 1 wherein inhibiting neuronal cell death comprises preventing neuronal cell death under conditions where cell death may occur.

4. The method of claim 1, wherein the inhibition of neuronal cell death is inhibition of cell death by necrosis.

5. The method of claim 1, for inhibiting neuronal cell death under conditions where cell death may occur.

6. The method of claim 5, wherein the conditions where cell death may occur is stress-induced neuronal cell death.

7. The method of claim 6, wherein the stress-induced neuronal cell death is pH-stress induced neuronal cell death.

8. The method of claim 7 wherein the pH stress induced neuronal cell death is alkalosis-stress induced neuronal cell death.

9. The method of claim 8, wherein the alkalosis-stress induced neuronal cell death is at a pH greater than 7.4.

10. The method of claim 9, wherein the pH is from 8.0 and 9.0.

11. The method of claim 10, wherein the pH is from 8.0 and 8.4.

12. The method of claim 5, wherein the conditions where cell death may occur is physiological trauma.

13. The method of claim 12, wherein the physiological trauma is selected from the group consisting of: hypoxia, injury, infection, cytokine deprivation, carcinogenic agents and cancer.

14. The method of claim 12, wherein the physiological trauma is a result of neurodegenerative disease.

15. The method of claim 14, wherein the neurodegenerative disease is selected from the group consisting of: Alzheimer's, Parkinson's, Huntington's, Multiple Sclerosis and brain ischemia.

16. The method of claim 12, wherein the physiological traumas is selected from the group consisting of: hypothermia, hypoxia, acute ischemia, hypoxia-ischemia, respiratory alkalosis, metabolic alkalosis and brain alkalosis.

17. The method of claim 12, wherein the physiological trauma is traumatic injury to the brain or spinal cord.

18. The method of claim 17, wherein cell death is a result of secondary energy failure post the physiological trauma.

19. The method of claim 1, wherein the neuronal cell is a hypothalamic cell.

20. A method of treating a neuronal condition associated with alkaline neuronal cell pH conditions comprising administering to a patient in need thereof an effective amount of TCAP, pharmaceutical acceptable salt or ester thereof or obvious chemical equivalent thereof.

21. A method for increasing neuronal cell proliferation under conditions of neutral pH or acidosis comprising administering to a patient in need thereof an effective amount of TCAP, pharmaceutical acceptable salt or ester thereof or obvious chemical equivalent thereof.

22. The method of claim 21, wherein pH conditions are pH of 7.4 or less.

23. The method of claim 21 wherein the pH conditions are 6.8 or less.

24. The method of claim 22, wherein the pH conditions are from 6.8 to 7.4
